SEQUENCE LISTING (4 sequences)

SEQ ID NO: 1 - protein, 12 aa, synthetic construct
  source 1..12: note = GE11
YHWYGYTPQNVI

SEQ ID NO: 2 - protein, 12 aa, synthetic construct
  source 1..12: note = TfR-T12
THRPPMWSPVWP

SEQ ID NO: 3 - protein, 15 aa, synthetic construct
  source 1..15: note = 15K
IRKAHCNISRAKWND

SEQ ID NO: 4 - protein, 15 aa, synthetic construct
  source 1..15: note = 15D
IRKAHCNISRADWND

(19) United States
(12) Patent Application Publication
(10) Pub. No.: US 2025/0228786 A1
(43) Pub. Date: Jul. 17, 2025

(54) METHOD FOR PRODUCING DELIVERY VESICLES

(71) Applicants: Deutsches Krebsforschungszentrum Stiftung des öffentlichen Rechts, Heidelberg (DE); Hepione Therapeutics Inc., New York, NY (US); Panosome GmbH, Heidelberg (DE)

(72) Inventors: Fotini Papavasiliou-Stebbins, Heidelberg (DE); Erec Stebbins, Heidelberg (DE); Joseph Verdi, New York, NY (US); Dimitra Stamkopoulou, Heidelberg (DE); Jose Paulo Lorenzo, Heidelberg (DE); Albina Waithaka, Heidelberg (DE); Riccardo Pecori, Heidelberg (DE); Beatrice Casati, Heidelberg (DE); Annette Arnold, Heidelberg (DE)

(21) Appl. No.: 18/847,596

(22) PCT Filed: Mar. 15, 2023
(86) PCT No.: PCT/EP2023/056630

§ 371 Date: Sep. 16, 2024

(30) Foreign Application Priority Data
Mar. 15, 2022 (EP) 22162092.5

(51) Int. Cl. A61K 9/50; A61K 47/68; A61K 47/69; C12P 21/00

(52) U.S. Cl. CPC A61K 9/5068; A61K 47/6835; A61K 47/6913; C12P 21/005

(57) ABSTRACT

The present invention concerns the development of vesicles that could be used for generation of vaccines or as compound delivery vehicles. More specifically, the invention relates to a method for preparing a vesicle comprising the steps of: providing recombinant Trypanosoma brucei cells expressing sortaggable VSG, treating said cells in hypotonic solution in the presence of at least one protease inhibitor until the cells are lysed, isolating the cellular membranes from the solution, suspending the isolated membranes previously obtained in a isotonic solution, treating the suspended cellular membranes obtained in the previous step with sonication in order to obtain a vesicle suspension, removing aggregated membranous debris from the vesicle suspension previously obtained, separating the vesicle suspension into populations of vesicles, and providing vesicles from a population of vesicles which is characterized by the following parameters: (i) having a single predominant protein revealed after Coomassie staining an SDS PAGE that has an apparent molecular weight of 55 to 60 kDa, (ii) having a spherical appearance in electron micrographs and (iii) exhibiting a homogenous surface structure in electron micrographs. Moreover, the present invention also relates to a vesicle comprising sortaggable VSG characterized by the aforementioned parameters as well as such a vesicle for use in treating and/or preventing a disease or medical condition or as a compound delivery vesicle, preferably, drug delivery vehicle, more preferably, nucleic acid delivery vesicle. Finally, the invention contemplates a kit for carrying out the method of the present invention comprising recombinant Trypanosoma brucei cells expressing sortaggable VSG and at least one agent for carrying out the method of the present invention or a kit comprising the vesicle of the present invention.

The present invention concerns the development of vesicles that could be used for the generation of vaccines or as compound delivery vehicles. More specifically, the invention relates to a method for preparing a vesicle comprising the steps of: providing recombinant Trypanosoma brucei cells expressing sortaggable VSG, treating said cells in hypotonic solution in the presence of at least one protease inhibitor until the cells are lysed, isolating the cellular membranes from the solution, suspending the isolated membranes previously obtained in a isotonic solution, treating the suspended cellular membranes obtained in the previous step with sonication in order to obtain a vesicle suspension, removing aggregated membranous debris from the vesicle suspension previously obtained, separating the vesicle suspension into populations of vesicles, and providing vesicles from a population of vesicles which is characterized by the following parameters: (i) having a single predominant protein revealed after Coomassie staining an SDS PAGE that has an apparent molecular weight of 55 to 60 kDa, (ii) having a spherical appearance in electron micrographs and (iii) exhibiting a homogeneous surface structure in electron micrographs. Moreover, the present invention also relates to a vesicle comprising a sortaggable VSG characterized by the aforementioned parameters as well as such a vesicle for use in treating and/or preventing a disease or medical condition or as a compound delivery vesicle, preferably, drug delivery vehicle, more preferably, nucleic acid delivery vesicles. Finally, the invention contemplates a kit for carrying out the method of the present invention comprising recombinant Trypanosoma brucei cells expressing a sortaggable VSG and at least one agent for carrying out the method of the present invention or a kit comprising the vesicle of the present invention.

Despite its promise, the field of gene therapy has yet to overcome an all-important dilemma: how to deliver the therapy of interest to a specific target site with efficacy. Conventional drugs can be fused to cell-type specific ligands or antibodies. However, this unfortunately does not apply to nucleic acids because of poor pharmacokinetics, sensitivity to enzymatic destruction, and large size. To realize the promise of gene therapy, novel tools are required.

Adenoviruses or AAV: Efficient targeting but poor loading (also: immunogenicity). The majority of delivery systems employed by the field involve viruses-which are essentially nucleic acid packaging systems coated in a protein responsible for driving the uptake of that genetic information into a specific target cell by binding to a surface receptor. Multiple viruses have been trialed for gene therapy purposes, often with adverse effects (e.g., retroviruses with “off-target” oncogenic effects). Adeno-associated virus (AAV), manipulated such that it is rendered non-replicative, has a specific advantage in that virions can acquire different tropism based on the exact composition of their capsid (Kuzmin et al., 2021). The combination of AAV serotype (and thus tissue targeting) with the specific promoter driving expression (e.g., of an encapsidated RNA) are hoped to achieve tissue-restricted delivery. The substantial interest in this approach has been boosted by the recent approval of two AAV-based gene therapies by the US FDA.

Despite its promise, however, there are several issues associated with AAV-based gene therapy approaches. First, 30-60 percent of humans have preexisting antibodies to some AAV serotypes, leading to a loss of efficacy even with the first dose (Kruzik et al., 2019). Second, the packaging efficiency of recombinant AAV is limited to nucleic acids under 5 kb in size (Wu et al., 2010). Third, AAVs are usually produced recombinantly by transfection, posing issues of scalability (especially as clinical trials report dosing of 1012-1013 recombinant (r) AAV genome copies per kg of body weight for liver transduction gene therapy (Miesbach et al., 2018) thus reaching acceptable cost limits of current production techniques). Finally, it is of note that ˜90% of rAAV capsids are “empty” and, in addition to inefficacy, can serve as a potential source of toxicity (Gao et al., 2014); though some claim that empty particles can also be used as decoys to suppress pre-existing immunity.

LNPs: highly efficient loading and delivery but poor targeting. These issues and additional challenges with other viruses have led many to propose nanoparticle-based methods for gene therapy delivery. Nanoparticles can be broadly distinguished into polymer based and lipid-based nanoparticles (LNPs). Polymer based nanoparticles depend on the synthesis of a backbone (e.g. polyamide or poly-propyleneimine backbone) followed by selection of size, charge, and composition-making them versatile in structure and properties. LNPs can be formulated by combining different biomaterials with cholesterols, lipid-PEG compounds, and other lipids, and are currently broadly used e.g., in the context of Sars-CoV-2 vaccines. In both cases, libraries of resulting nanoparticles (generated by varying the molar ratio of the different constituents) have demonstrated some degree of tissue selectivity (Lokugamage et al., 2018). While this approach is promising, it relies on the (unknown and not understood) similarities that might determine translatability of targeting between human and mouse—and therefore the ability to use model systems to understand nanoparticle tropism in human. The bottom line is that LNPs are not targetable.

However, there is a need for a delivery system that is capable of being efficiently loaded and that is highly efficient as regards targeting as well since many pharmaceutical or biotechnological applications require both.

The technical problem underlying the present invention may be seen as the provision of means and methods for complying with the aforementioned needs. The technical problem is solved by the embodiments characterized in the claims and herein below.

Thus, the present invention relates to a method for preparing a vesicle comprising the steps of:
a) providing recombinant Trypanosoma brucei cells expressing a VSG, preferably, a sortaggable VSG;
b) treating said cells in hypotonic solution in the presence of at least one protease inhibitor until the cells are lysed;
c) isolating the cellular membranes from the solution of step b);
d) suspending the isolated membranes obtained in step c) in an isotonic solution;
e) treating the suspended cellular membranes obtained in step d) with sonication in order to obtain a vesicle suspension;
f) removing aggregated membranous debris from the vesicle suspension obtained in step e);
g) separating the vesicle suspension into populations; and
h) providing vesicles from a population of vesicles which is characterized by the following parameters: (i) having a single predominant protein revealed after Coomassie staining an SDS PAGE that has an apparent molecular weight of 55 to 60 kDa, (ii) having a spherical appearance in electron micrographs and (iii) exhibiting a homogenous surface structure in electron micrographs.

It is to be understood that in the specification and in the claims, “a” or “an” can mean one or more of the items referred to in the following depending upon the context in which it is used. Thus, for example, reference to “an” item can mean that at least one item can be utilized.

As used in the following, the terms “have”, “comprise” or “include” are meant to have a non-limiting meaning or a limiting meaning. Thus, having a limiting meaning these terms may refer to a situation in which, besides the feature introduced by these terms, no other features are present in an embodiment described, i.e. the terms have a limiting meaning in the sense of “consisting of” or “essentially consisting of”. Having a non-limiting meaning, the terms refer to a situation where besides the feature introduced by these terms, one or more other features are present in an embodiment described.

Further, as used in the following, the terms “preferably”, “more preferably”, “most preferably”, “particularly”, “more particularly”, “typically”, and “more typically” are used in conjunction with features in order to indicate that these features are preferred features, i.e. the terms shall indicate that alternative features may also be envisaged in accordance with the invention.

Further, it will be understood that the term “at least one” as used herein means that one or more of the items referred to following the term may be used in accordance with the invention. For example, if the term indicates that at least one item shall be used this may be understood as one item or more than one item, i.e. two, three, four, five or any other number. Depending on the item the term refers to the skilled person understands as to what upper limit the term may refer, if any.

The term “about” as used herein means that with respect to any number recited after said term an interval accuracy exists within in which a technical effect can be achieved. Accordingly, about as referred to herein, preferably, refers to the precise numerical value or a range around said precise numerical value of +20%, preferably +15%, more preferably +10%, or even more preferably +5%.

The term “comprising” as used herein shall not be understood in a limiting sense. The term rather indicates that more than the actual items referred to may be present, e.g., if it refers to a method comprising certain steps, the presence of further steps shall not be excluded. However, the term also encompasses embodiments where only the items referred to are present, i.e. it has a limiting meaning in the sense of “consisting of”.

The method of the invention may consist of the aforementioned steps or may comprise further steps, such as steps for producing recombinant Trypanosoma brucei cells expressing a VSG, preferably, a sortaggable VSG or steps aiming at sortagging or coupling desired compounds to the VSG by sortase or chemical reactions. Moreover, there might be additional steps within or between the steps recited above. For example, step a) may also comprise sortagging or coupling desired compounds to the VSG by sortase or chemical reactions. Alternatively, sortagging or coupling may be carried out after providing the vesicles, i.e. after step g).

In step a) of the method of the present invention, recombinant Trypanosoma brucei cells expressing a VSG, preferably, a sortaggable VSG are provided.

The term “recombinant Trypanosoma brucei cells” refers to a single cell parasitic organism. Trypanosoma brucei is a pathogenic organism responsible for the sleeping sickness. Trypanosoma brucei as referred to herein encompasses all subspecies such as Trypanosoma brucei brucei, Trypanosoma brucei gambiense and Trypanosoma brucei rhodesiense. Trypanosoma brucei cells can be genetically modified and, thus, allow for the generation of recombinant organisms. Trypanosoma brucei exhibits on its surface glycoprotein coat comprising the variant surface glycoprotein (VSG).

The term “VSG” as used herein refers to a glycoprotein belonging into a class of glycoproteins of approximately 60-kDa as a monomer which may be found as monomers, dimers, trimers or multimers densely located on the outer membrane in order to form a surface coat. VSG dimers make up approx. 90% of all cell surface proteins in trypanosomes. VSGs are highly immunogenic and an immune response raised against a specific VSG coat will rapidly kill trypanosomes expressing this variant. However, with each cell division there is a chance that the progeny will switch expression of a VSG gene to another to change the VSG that is being expressed and, thus, escape immunity. Only one VSG gene is expressed at a time. VSG expression is ‘switched’ by homologous recombination induced by double-strand breaks of a silent basic copy gene from an array (directed by homology) into the active telomerically-located expression site. VSG annotation, protein sequences and gene sequences are derivable from public databases such as www.ensemble.org. A collection of VSGs of Trypanosoma brucei brucei (Lister 427 strain) is published in Cross 2014, Mol. Biochem. Parasitol. 195 (1): 59-73. Preferred VSGs in context of the present invention are Trypanosoma brucei VSGs, more preferably VSG2, VSG3 ILTat1.24, VSG11, VSG13, VSGsur, VSG1954, or VSG531. A preferred VSG according to the invention which is characterized by an N-terminus which is located 3-dimensionally within the VSG at a position which is, when the VSG is present within a VSG coat, for example, on a trypanosome cell, located sufficiently close to the accessible outer coat-surface such that the addition of a linker sequence, preferably not more than 100 amino acids, preferably not more than 50 amino acids, and most preferably not more than 20 amino acids in length, allows for modification of the so extended or not extended N-terminus of the VSG protein. Preferably, the insertion may be immediately downstream of the signal peptide cleavage site.

Moreover, the recombinant Trypanosoma brucei cells may be genetically engineered to express more than one type of VSG, so that vesicles obtained from such genetically engineered Trypanosoma brucei cells by the method of the present invention shall comprise two or more different types of VSGs on the surface of the vesicle.

Moreover, the recombinant Trypanosoma brucei cells, preferably, lack GPI phospholipase C. The genetic deletion of the endogenous glycophosphatidylinositol phospholipase C (GPI-PLC), i.e. the enzyme that “sheds” VSG off the surface of dying cells, is crucial to generating T. brucei that can be used, e.g., as antigen display platform, because unless GPI-PLC is removed from the genome, any form of inactivation of the trypanosome cells (e.g. UV-irradiation) will also lead to the disintegration of the VSG coat and of the cell itself. Once VSGs are shed due to the action of GPI-PLC, the VSG coat disintegrates and the cells lyse.

The term “sortaggable” as used herein refers to a VSG that can be linked to another polypeptide or peptide by sortase activity. Preferably, the VSG will comprise a sortase donor or a sortase acceptor amino acid sequence. Typically, said sortase donor or acceptor sequence may be introduced into the VSG by genetic engineering. For example, the said sortase donor or acceptor sequence may be comprised in a peptide that will be fused to the VSG. Alternatively, a suitable endogenous sequence of the VSG may be genetically modified such that it becomes a sortase donor or acceptor sequence.

A sortase as used herein, refers to a protein having sortase activity, i.e., an enzyme able to carry out a transpeptidation reaction conjugating the C-terminus of a protein to the N-terminus of a protein via transamidation. The term includes full-length sortase proteins, e.g., full-length naturally-occurring sortase proteins, fragments of such sortase proteins that have sortase activity, modified (e.g., mutated) variants or derivatives of such sortase proteins or fragments thereof, as well as proteins that are not derived from a naturally occurring sortase protein, but exhibit sortase activity. Those of skill in the art will readily be able to determine whether or not a given protein or protein fragment exhibits sortase activity, e.g., by contacting the protein or protein fragment in question with a suitable sortase substrate under conditions allowing transpeptidation and determining whether the respective transpeptidation reaction product is formed. Suitable sortases will be apparent to those of skill in the art and include, but are not limited to, sortase A, sortase B, sortase C, and sortase D type sortases. Suitable sortases are described, for example, in Dramsi 2005, Res. Microbiol. 156 (3): 289-97, Comfort 2004, Infect Immun., 72 (5): 2710-22, Chen 2011, Proc Natl Acad. Sci. USA. July 12; 108 (28): 11399, and Pallen 2001, TRENDS in Microbiology, 2001, 9 (3), 97-101. Moreover, the present invention encompasses embodiments relating to a sortase A from any bacterial species or strain. Those of skill in the art will appreciate that any sortase and any sortase recognition motif can be used in some embodiments of this invention, including, but not limited to, the sortases and sortase recognition motifs described in WO2010/087994, WO2011/133704, and WO 2020/84072.

The sortase substrates are amino acid sequences that can be utilized in a sortase-mediated transpeptidation reaction. Typically, a sortase utilizes two substrates, a substrate comprising a C-terminal sortase recognition motif, and a second substrate comprising an N-terminal sortase recognition motif and the transpeptidation reaction results in a conjugation of both substrates via a covalent bond. In context of the invention the “C-terminal sortase recognition motif” is also referred to as “sortagging donor sequence”, whereas the term “N-terminal sortase recognition motif” is referred to as “sortagging acceptor sequence”. Preferably, the C-terminal and N-terminal recognition motifs are comprised in different amino acid sequences, for example, one N-terminally of the VSG, and the other linked to the immunogen such that there is a free carboxyl group at the end of the sortagging donor site. Some sortase recognition motifs are described herein and additional suitable sortase recognition motifs are well-known to those of skill in the art. Sortase recognition motifs will be apparent to those of skill in the art. A sortase substrate may comprise additional moieties or entities apart from the peptidic sortase recognition motif.

For example, a sortase substrate may comprise an LPXTG/A motif, the N-terminus of which is conjugated to any agent, (e.g. a peptide or protein, a small molecule, a binding agent, a lipid, a carbohydrate, or a detectable label). Similarly, a sortase substrate may comprise an oligoglycine (G1-5) motif or oligoalanine motif, preferably G3 or G5, the C-terminus of which is conjugated to any agent, e.g., a peptide or protein, a small molecule, a binding agent, a lipid, a carbohydrate, or a detectable label. Accordingly, sortase substrates are not limited to proteins or peptides but include any moiety or entity conjugated to a sortase recognition motif.

The VSG shall be, preferably, “sortaggable”, i.e. it shall be a sortase substrate and, thus, comprise a sortase donor sequence or sortase acceptor sequence. An example of a sortaggable VSG can be derived from Pinger 2017, Nat Commun. 8 (1): 828. Depending on whether the VSG comprises a sortase acceptor or donor, a molecule to be linked by sortagging to the VSG shall comprise the complement, i.e. in case of the sortase acceptor being comprised by the VSG, it shall comprise a sortase donor or in case of a sortase donor being comprised by the VSG, it shall comprise a sortase acceptor.

It will be understood that said VSGs may also be modified chemically. Preferably, said VSGs may be modified by chemical coupling reactions in order to couple desired molecules such as targeting compounds specified elsewhere herein. Typically, targeting compound may be covalently linked, preferably via a linker, to the N-terminus of the VSG. Targeting compounds may be linked to the VSG by any techniques known in the art, including any chemical reaction. Preferably, click-chemistry or other cross-linking may be used. Click-chemistry in context of the invention shall refer to chemistry tailored to generate covalent bonds quickly and reliably by joining small units comprising reactive groups together. Any variants of such approaches may be used to connect the targeting compound to the VSG in accordance with the invention. In general, the linking of the VSG to the targeting compound may include the use of any linking means or linker, which in context of the herein disclosed invention refers to the means by which the VSG and the targeting compound are linked or connected to form a modified VSG. The one or more linkers or linking means for linking the VSG and the targeting compound may be any structurally suitable means to connect the two. Exemplary linkers include the use of one or more amino acids which may be used to form a peptide, in some embodiments having a modified peptide backbone, a small chemical scaffold, a biotin-streptavidin, an organic or inorganic nanoparticle, a polynucleotide sequence, peptide-nucleic acids, an organic polymer, or an immunoglobulin Fc domain. The means for linking can comprise covalent and/or noncovalent bonds. The one or more linkers can include various sequences or other structural features that provide various functions or properties. For example, the one or more linkers can contain structural elements to allow the VSG to be derivatized.

Preferably, the recombinant Trypanosoma brucei cells expressing a VSG, preferably, a sortaggable VSG are provided in purified or partially purified form. Typically, the said cells shall be free or essentially free of any cultivation media. Preferably, such a preparation of recombinant Trypanosoma brucei cells expressing a VSG, preferably, a sortaggable VSG can obtained by centrifugation allowing for separation of media and cells and resuspension of the cells in a suitable solvent such as the hypotonic solution to be used for carrying out the subsequent step b). In case that preparation of recombinant Trypanosoma brucei cells expressing sortaggable VSG shall be stored, an isotonic solution which allows for storage of the cells may be used. Suitable solutions are well-known to the person skilled in the art. Particular preferred techniques for providing the recombinant T. brucei cells are described in the accompanying Examples, below. These techniques may encompass centrifugation of the cells in order to obtain a pellet of cells as well carrying out one or more washing steps.

More preferably, the T. brucei cells as grown in cell culture medium, preferably, standard HMI9 with 10% FBS are centrifuged at about 2,000 g for about 20 min at about room temperature. Preferably, the pelleted cells are washed twice in phosphate buffered saline (PBS). Such washed cells are subsequently subjected to lysis according to step b) of the method of the invention. Typically, about 5 billion cells are provided for further prosecution.

In step b) of the method of the invention, said cells, i.e. the recombinant Trypanosoma brucei cells expressing a VSG, preferably, a sortaggable VSG, are treated in hypotonic solution in the presence of at least one protease inhibitor until the cells are lysed.

The term “hypotonic solution” as used herein refers to a solution that shall be hypotonic with respect to the cytoplasm of the Trypanosoma cells. Suitable hypotonic solutions are well-known to the skilled person. Typically, a hypotonic solution may be deionized water or a buffer solution with low salt content. The water molecules in the hypotonic solution will move into the inside of the cells, i.e. into the cytoplasm, due to osmosis. As a result, the volume of the cytoplasm will increase and destroy the cell membrane such that the cell membrane is disrupted and form several pieces of isolated cell membranes. Preferably, deionized water is used as a hypotonic solution.

Treatment shall be carried out for a time and under conditions sufficient to allow for osmotic disrupting the cell membrane of the Trypanosoma cells as described before. Preferably, treatment is carried out for a time between about 30 min and about 90 min minutes, more preferably, about 45 min to about 60 min, most preferably about 60 min. More specifically, the cells are treated for 10 min, centrifuged and the pellet is resuspended. These steps may be repeated several times to sum up to the aforementioned treatment times. Furthermore, treatment may also encompass gentle or ridged shaking of the cells. The temperature applied during said treatment using hypotonic solution is typically a temperature between about 4° C. and about 37° C., more typically, at a temperature of about 4° C.

The treatment is also performed in the presence of at least one protease inhibitor. The said at least one protease inhibitor shall prevent protease activity which may degrade the VSG proteins of the VSG coat present on the membranes. Suitable protease inhibitors are known to the person skilled in the art. In particular, it is envisaged to use a mixture of several different protease inhibitors. Preferably, the HALT protease inhibitor composition as referred to herein comprises at least a serine protease inhibitor, an amino-peptidase inhibitor, a cysteine protease inhibitor, a metalloprotease inhibitor, a serine and cysteine protease inhibitor and/or an aspartic acid protease inhibitor. More preferably, it comprises AEBSF, aprotinin, bestatin, E-64, EDTA, leupeotin, and pepstatin A. More preferably, said mixture of protease inhibitors is the commercially available HALT protease inhibitor composition (ThermoFisher Scientific, Inc.).

Lysis of the cells typically results in disrupted cellular membranes of the recombinant Trypanosoma brucei cells expressing sortaggable VSG, also referred to as “cellular membranes”. Said membranes may be variable in size and shall comprise, preferably, intact VSGs. The degree of lysis may be visualized by applying a dye for living cells. Particular preferred lysis conditions are described in the accompanying Example, below.

More preferably, the pelleted T. brucei cells provided in step a) of the method of the invention are suspended in ice-cold deionized water for lysis in the presence of the HALT protease composition.

In step c) of the method of the invention, the cellular membranes are isolated from the solution obtained when carrying out step b).

The isolation of the cellular membranes from residual material of the lysed cells can be achieved by techniques well-known in the art. Preferably, the cellular membranes are isolated by centrifugation, more preferably, as described in the accompanying Examples, below. Alternatively, the cellular membranes may be obtained in isolated form by filtration, affinity purification using, e.g., antibodies against VSGs, size exclusion chromatography, differential centrifugation based on size/density, ion-exchange chromatography, gel filtration chromatography, osmotic enrichment using semi-permeable membranes or electrophoretic separation techniques. The aforementioned techniques for isolating the cellular membranes may be used alone or in combination in order to achieve a higher degree of purity of the cellular membranes. Moreover, the cellular membranes may be subjected to one or more washing steps.

Typically, the isolation of the cellular membranes is carried out at a temperature as described for step b), above, and, more preferably, at about 4° C. More preferably, the cellular membranes are isolated by centrifugation at about 10,000 g for about 10 min at about 4° C. Subsequently, the pellet is, preferably re-suspended in ice-cold deionized water and the centrifugation is repeated under the same conditions.

In step d) of the method of the invention, the isolated membranes obtained in step c) are suspended in an isotonic solution.

The term “isotonic solution” as used herein refers to a solution that shall be isotonic with respect to the cytoplasm of the Trypanosoma cells. Suitable isotonic solutions are well-known to the skilled person. Typically, an isotonic solution may be a buffer solution with medium (physiological) salt content such as a phosphate-, HEPES- or Tris-based buffer. Preferably, 20 mM HEPES, 150 mM NaCl₂. pH 8.0 is used as a hypotonic solution.

In step e) of the method of the invention, the suspended cellular membranes obtained in step d) are treated with sonication in order to obtain a vesicle suspension.

Sonication can be carried out by applying constant or pulsar sonic for a time sufficient to allow formation of vesicle structures in the solution. Typically said time is between about 3 min to about 10 min, more preferably, between about 4 min and about 6 min, most preferably, about 5 min. More preferably, sonication is carried out as described in the Examples below, i.e. for 5 min, at 40% duty using a Bandelin Sonoplus machine with pulsar on ice. This treatment, preferably, results in sheering the large cellular membranes such that smaller vesicle structures are obtained including vesicles with an average diameter of about 250 nm.

In step f) of the method of the invention, aggregated membranous debris are removed from the vesicle suspension obtained in step e).

Preferably, the aggregated membranous debris are removed from the vesicle suspension obtained in step e) by centrifugation and filtration. Preferably, filtration is carried out by using a 0.45 uM filter.

Aggregated membrane debris, typically having a size of about 750 nm or more, are removed by centrifugation, preferably, carried out at about 2,000 g for about 5 min at about 4° C. Subsequently, a filtration step shall be carried out. Preferably, a filter with a sieve in the micro-meter range is used. Pressure may be applied by well-known methods and typically by using a syringe. Preferably, aggregated membranous debris are removed from the vesicle suspension obtained in step e) by centrifugation and filtration as described in the accompanying Examples, below. More preferably, the suspension comprising the vesicles and the aggregated membrane debris is centrifuged at 2,000 g for 5 min at 4° C. such that remaining large aggregated pelletable membrane debris and intact cells, if any, are pelleted. Subsequently, the supernatant shall be filtered through a 0.45 uM filter using a 10 ml syringe.

Each of the aforementioned steps relating to sonication, centrifugation and/or filtration may be, preferably, repeated at least once in accordance with the present invention.

In step g) of the method of the present invention, the vesicle suspension is separated into populations.

Separation into populations of vesicles of about similar size can be achieved by various means.

Preferably, separation as referred to herein is separation by size. More preferably, size exclusion chromatography, such as gel filtration or CaptoCore chromatography, may be used. Alternatively, centrifugation based techniques, such as differential centrifugation, centrifugation with cesium or sucrose gradients or ultracentrifugation, may be applied. The skilled person is well aware of how such separation by size can be achieved.

Gel filtration of the vesicle suspension according to the present invention as referred to in the context of the present invention may be carried out by chromatographic method in which particles or molecules in solution are separated by their size. Typically, an aqueous solution, i.e. a mobile phase, is used to transport the sample through a stationary phase (gel) which may be present, e.g., in a column. The chromatography column is packed with fine, porous beads which are composed of polymers, preferably, dextran polymers (Sephadex), agarose (Sepharose), or polyacrylamide (Sephacryl or BioGel P). The polymer allows large particles or molecules to pass through the column rather quickly whereas smaller molecules may enter spaces formed by the polymer and thus may need longer to pass the column. Preferably, for gel filtration Sephacryl S-500 26/60 or Superose 6 columns may be used. Preferably, gel filtration may be carried out as described in the accompanying Examples, below.

CaptoCore chromatography as referred to in the context of the present invention refers to a purification method for purifying and polishing viruses and other large biomolecules in flowthrough mode. Typically, the chromatography media (resins) comprise beads (core beads) having a non-functionalized outer layer (without ligand) and functionalized core with an attached ligand. The inert outer layer of cross-linked agarose prevents viruses and other large entities with a molecular mass greater than approximately 400.000 (CaptoCore 400) or 700.000 (CaptoCore 700) from entering the core of the bead, while small molecules penetrate the beads where they are captured. The excluded target molecules are collected in the flowthrough. The functionalized core comprises an ocytlamine ligand in the core of the beads that is multimodal, i.e. is both hydrophobic and positively charged. This ligand binds strongly various contaminants over a wide range of pH and salt concentrations. CaptoCore chromatography can be used at single or multiple points during preparation of the delivery particles of the present invention. For example, CaptoCore chromatography can be applied prior to or after purification of the vesicles, after sortagging or prior to application of the vesicles.

More preferably, the vesicle suspension obtained after filtering in step f) can be subjected to gel filtration chromatography on a 20 mM HEPES, 150 mM NaCl2, pH 8-equilibrated Sephacryl S-500 26/60 column (FIG. 1A) in order to remove small debris and to isolate the largest vesicles. The latter of which account for the majority of the contents of the preparation and these vesicles are henceforth also referred to as “nanoVASTs”.

Preferably, the populations of vesicles may be obtained by using chromatographic techniques other than those based on size exclusion. More preferably, ion-exchange or immune affinity chromatography may be applied. The skilled person is well aware of how such techniques may be carried out.

In step h) of the method of the invention, vesicles are provided from a population of vesicles obtained in step g). The vesicles obtained by the method of the present invention (i.e. the nanoVASTs) may be subjected to a variety of characterizations, including SDS-PAGE for protein purity and dynamic light scattering for vesicle size and uniformity determinations (FIGS. 1B and C). Said provided vesicles are characterized by the following parameters: (i) having a single predominant protein revealed after Coomassie staining an SDS PAGE that has an apparent molecular weight of 55 to 60 kDa (ii) having a spherical appearance in electron micrographs and (iii) exhibiting a homogenous surface structure in electron micrographs. SDS PAGE as referred to herein is preferably carried out as described in the accompanying Examples, below. In particular, nanoVAST samples are diluted into standard SDS-PAGE denaturing sample buffer prior to separation on 10% tris-glycine polyacrylamide gels. The gels are then either Coomassie stained to observe the variety of proteinaceous constituents, or subjected the western blot analyses to identify the VSG band specifically. Standard transmission electron micrographs are, preferably, taken using the following setup and conditions, vesicles from purified preparations are adsorbed onto glow discharged carbon coated copper grids (mesh-size 300), washed in double distilled water and negatively stained with 1% aqueous uranyl acetate. Micrographs were taken with a Zeiss EM912 at 80 kV (Carl Zeiss, Oberkochen, Germany) using a slow scan CCD camera (TRS, Moorenweis, Germany) and resulting pixel-size of 1.2 or 0.6 nm (depending on nominal magnification 10 kx versus 20 kx, respectively). Cryo-electron micrographs are, preferably, taken using the following setup and conditions: Samples are loaded onto plasma-cleaned UltrAuFoil grids (200 mesh; pore size 2 uM by 2 uM), then blotted for 2 seconds and plunge frozen using an FEI Vitrobot Mark IV. The grids are imaged using a Talos Artica microscope.

Preferably, said population of vesicles is further characterized by an average diameter within the range of about 50 nm to about 500 nm, preferably, about 150 nm to about 250 nm as determined by dynamic light scattering analysis. Dynamic light scattering analysis is, preferably, carried out as described in the Example 1, below. In particular, a ZetaSizer Nano (Malvern Playtical) was used for the measurement and the accompanying Zetasizer software was used for the analysis of the vesicles. 20 ul of the sample were diluted in 1 ml of PBS and placed into disposable polystyrene cuvettes and then measured.

Advantageously, it has been found in the studies underlying the present invention that artificial vesicles can be generated from T. brucei cell membranes that allow for efficient loading as well as excellent targeting and, thus, avoid the deficiencies of other delivery systems known in the prior art. The nanoVAST vesicles consist of a phospholipid bilayer (which would protect the cargo from e.g. nucleases or proteases), that could be decorated with targeting molecules such as surface receptors or ligands. Such “synthetic extracellular vesicles” (or EVs) would essentially mimic naturally occurring exosomes in form and function: they would be (i) naturally able to shuttle genetic information between distant cells (Bobis-Wozowicz et al., 2015) (ii) they would be stable in body fluids and (iii) they would be stored for extended periods.

Currently, natural EVs are not produced in large enough quantities to be useful. However, the loading, targeting and overall efficiency of synthetic EVs in the prior art remains low (Almeida et al., 2020). The nanoVAST vesicles are centered on a semi-synthetic EV whose main backbone can be produced in large quantities and which can be efficiently loaded with protein as well as nucleic acid cargo. In addition, nanoVAST vesicles can be enzymatically fused to receptor binding ligands specific to individual cell types, ensuring targeted delivery from the get-go. nanoVAST vesicles are cheap to manufacture, are scalable, and retain the best qualities of the viral system (cell type-specific targeting and convenient packaging) while avoiding excessive immunogenicity or oncogenic potential.

The similarities of the nanoVAST technology to naturally occurring exosomes (nature's own inter-tissue, long range delivery service), the feasibility of loading nanoVAST vesicles with payload of interest, and the ability to derivatize them with rationally designed “tags” (e.g., antibodies) that target specific cell types, will be a great improvement for the field of precision payload delivery.

The following embodiments are preferred embodiments of the method of the invention. The explanations of the terms made above apply mutatis mutandis.

In a preferred embodiment of the method of the invention, recombinant Trypanosoma brucei cells lack GPI phospholipase C.

In a preferred embodiment of the method of the invention, said at least one protease inhibitor is the HALT protease inhibitor composition.

In a preferred embodiment of the method of the invention, said method further comprises after step c) and prior to step d) the steps of:
treating said cells in deionized water solution; and
isolating the cellular membranes from said solution.

In a preferred embodiment of the method of the invention, said isotonic solution is a buffer comprising 20 mM HEPES, 150 mM NaCl₂, pH 8.

In a preferred embodiment of the method of the invention, said sonication in step e) is carried for 5 minutes, 40% duty, with pulsar.

In a preferred embodiment of the method of the invention, said removing aggregated membranous debris from the vesicle suspension in step f) is carried out by filtration in step f) is carried out by using a 0.45 uM filter.

In a preferred embodiment of the method of the invention, said separation in step g) comprises gel filtration that is carried out through gel filtration chromatography on a 20 mM HEPES, 150 mM NaCl₂, pH 8-equilibrated Sephacryl S-500 column or through a Superose 6 column.

In a preferred embodiment of the method of the invention, said method further comprises introducing a cargo agent of interest into the vesicles provided in step h). Preferably, said introducing comprises the steps of:
a) suspending the vesicles in transfection buffer comprising an excess of cargo agent of interest;
b) carrying out electroporation; and
c) purifying loaded vesicles after electroporation.

The cargo agent of interest may also be introduced into the vesicles by freeze-drying, said introducing comprising the steps of:
a) freeze-drying the vesicles in the presence of the cargo;
b) resuspending the freeze-dried vesicles and cargo in a resuspension buffer;
c) mixing the resuspension mixture; and
d) purifying the loaded vesicles.

More preferably, said cargo agent of interest is selected from the group consisting of: small molecule drugs, peptides, proteins, and nucleic acid molecules.

The term “cargo molecule” as used herein refers to any compound that needs to be transported by the vesicle of the invention. Typically, such compounds are molecules that need to be transported in encapsulated form, e.g., compounds that shall enter cells but that shall not interact with extracellular structures in an organism or which are unstable in culture media or the like. Preferably, a cargo molecule according the present invention is selected from the group consisting of small molecule drugs, antibodies, aptamers, therapeutic proteins and peptides, such as cytokines, growth factors, hormones, and the like, nucleic acid molecules such as RNA or DNA, and detectable labels, such as dyes used for staining cells.

In a preferred embodiment, the cargo molecule that needs to be transported by the vesicle of the present invention is an RNA-editing molecule. More preferably, the RNA-editing molecule is an antisense nucleic acid, preferably, an antisense oligonucleotide. An “antisense oligonucleotide” (ASO) as used herein, refers to a single strand DNA and/or RNA molecule that is capable of interfering with DNA and/or RNA processing. Antisense oligonucleotides comprise a nucleic acid sequence, which is complementary to a specific RNA or DNA sequence.

Typically, an antisense oligonucleotide will bind, in a sequence-specific manner, to its respective complementary oligonucleotides, DNA, or RNA, thereby interfering with DNA and/or RNA processing. It is known to those skilled in the art that antisense oligonucleotides may interfere with mRNA processing through RNase H-mediated degradation, translational arrest, modulation of splicing or they may act through steric hindrance of proteins. Means and methods for the design and synthesis of antisense oligonucleotides are well-known in the art and include, for example, rational design, chemical modifications and design of antisense oligonucleotides containing locked nucleic acids (LNA) as well as solid-phase chemical synthesis. Antisense oligonucleotides can be chemically synthesized or expressed within the cell, for example, by introduction of a respective recombinant DNA construct. It will be understood by those skilled in the art that such a DNA construct may contain, in addition to a nucleic acid sequence encoding the antisense oligonucleotide, regulatory elements such as an enhancer, a constitutive or inducible promoter or a terminator.

Preferably, the cargo molecule according to the present invention is an antisense oligonucleotide capable of recruiting an RNA editing enzyme. More preferably, the RNA editing enzyme is adenosine deaminase acting on RNA (ADAR). The term “ADAR” refers to a double-stranded RNA specific adenosine deaminase, which catalyses the hydrolytic deamination of adenosine to inosine in double-stranded RNA (dsRNA), also referred to as A to I editing. Preferably, the ADAR is of human origin. The ADAR may be ADAR1, ADAR2 or ADAR3. The ADAR may be endogenous ADAR, recombinantly expressed in the target cell or delivered to the target cell.

The antisense oligonucleotide that needs to be transported by the vesicle of the invention may be part of an expression cassette. The term “expression cassette” refers to a distinct component of vector DNA consisting of a gene and regulatory sequence allowing expression in prokaryotic or eukaryotic cells or isolated fractions thereof. For example, the antisense oligonucleotides may be ligated into a nucleic acid expression construct (i.e. vector) under the transcriptional control of a cis-regulatory sequence suitable for directing constitutive or inducible transcription of the nucleotide sequence in a cell. Typically, an expression cassette comprises a promoter sequence, an open reading frame and a 3′ untranslated region (3′ UTR). The untranslated region may also contain a polyadenylation site in order to increase efficiency of translation.

In particular, the antisense oligonucleotide referred to before may be used for generating neoepitopes in immunopeptides associated with cancer and, preferably, is suitable for RNA editing, preferably, for the generation of a neoepitope in an immunogenic peptide in a cancer cell. The antisense oligonucleotide to be delivered as a cargo agent by the vesicle of the invention, thus, may be, preferably, capable of specifically hybridizing to the nucleic acid sequence of the mRNA which encodes at least one tumor epitope such that an ADAR can be recruited and can convert the said tumor epitope into the neoepitope. The neoepitope to be generated by RNA editing shall be, preferably, tumor specific and presented via HLA. To this end, the antisense oligonucleotide may be expressed from an expression construct, such as a vector. Preferably, said expression construct may also comprise an expression cassette for expressing an ADAR as mentioned before. More preferably, said vector used as an expression construct may be a viral vector such as a lentiviral vector. It will be understood that the vesicle in such a case shall be capable of targeting a cancer cell in which the neoepitope shall be generated.

In a preferred embodiment of the method of the invention, said method further comprises sortagging a targeting compound to the sortaggable VSG on the vesicles provided in step h). Preferably, said sortagging comprises the steps of:
a) treating the vesicles by sortase in the presence of targeting compound; and
b) purifying vesicles sortagged with the targeting compound.

The term “targeting compound” as used herein refers to a compound that allows for binding of the vesicle to a target molecule present on a target cell. Accordingly, a targeting compound may be any compound that is capable of interacting with the said target molecule. Typically, the targeting compound and the target molecule interact specifically. Preferably, the targeting compound is selected from the group consisting of: antibodies, nanobodies, aptamers, peptides, proteins, and small molecules. It will be understood that the sortaggable VSGs present on the surface of the vesicle of the invention may be coupled to one or more different targeting compounds as well. Further details on targeting compounds may be also found in WO2020/84072.

More preferably, said targeting compound is an antibody or nanobody recognizing a target molecule on a target cell.

The present invention also relates to a vesicle comprising a VSG, preferably, a sortaggable VSG characterized by the following parameters: (i) having a single predominant protein revealed after Coomassie staining an SDS PAGE that has an apparent molecular weight of 55 to 60 kDa, (ii) having a spherical appearance in electron micrographs and (iii) exhibiting a homogenous surface structure in electron micrographs. Preferably, the said vesicle has an average diameter of about 50 to about 500 nm, more preferably about 150 nm to about 250 nm, as determined by dynamic light scattering.

In a preferred embodiment of the vesicle of the invention, the vesicle is loaded with a cargo agent of interest, preferably, selected from the group consisting of: small molecule drugs, peptides, proteins, and nucleic acid molecules.

In a preferred embodiment of the vesicle of the invention, said vesicle is sortagged with a targeting compound, preferably, an antibody or nanobody recognizing a target molecule on a target cell.

In a preferred embodiment of the vesicle of the invention, said vesicle is obtainable by the method of the present invention.

The present invention also relates to a vesicle according to the present invention as defined before for use in treating and/or preventing a disease or medical condition. Moreover, the present invention also relates to a vesicle according to the present invention as defined before for use as a compound delivery vesicle, preferably, drug delivery vehicle.

The vesicle of the present invention may be used as a carrier for a drug and, thus, can be used for treating and/or preventing a disease. Suitable drugs are, preferably, selected from the group consisting of: small molecule drugs, antibodies, aptamers, therapeutic proteins and peptides, such as cytokines, growth factors, hormones, and the like, and nucleic acid molecules such as RNA or DNA. The skilled person is well aware of which diseases can be treated and/or prevented by administering encapsulated drugs and which are, therefore, suitable diseases to be treated and/or prevented by using drugs encapsulated in the vesicles of the invention. Moreover, depending on the disease, the vesicles may due to the presence of sortaggable VSG on its surface modified to allow for specific targeting of disease relevant cells, e.g., by sortagging of targeting antibodies or aptamers.

The vesicle of the present invention may also be used as a vaccine. To this end, the sortaggable VSGs on its surface may be coupled to an antigen or hapten by sortagging. The vesicles modified in such a way may be administered to a subject and cause immune reaction. Depending on the immunization scheme, such vesicles may be used for a priming immunization applied one or several times and/or boosting immunizations. Preferably, immunization using such vesicles may be carried out as described in WO2021/214043.

Yet the vesicle of the invention may be used as an in vitro delivery tool for drugs as specified above or other compounds to be introduced into cultured cells. Transfection techniques, e.g., may also apply the vesicles of the invention in order to introduce nucleic acid molecules into cultured cells.

The present invention also relates to a kit for carrying out the method of the present invention comprising recombinant Trypanosoma brucei cells expressing sortaggable VSG and at least one agent for carrying out the method of the present invention as defined before.

In a preferred embodiment of the kit of the invention, said at least one agent is selected from the group consisting of: deionized water, at least one protease inhibitor, preferably the HALT protease inhibitor composition, a buffer comprising 20 mM HEPES, 150 mM NaCl₂, pH 8, a 0.45 uM filter, a 20 mM HEPES, 150 mM NaCl₂, and sortase.

The present invention also relates to a kit comprising the vesicle of the present invention.

The following are particular preferred embodiments of the present invention.

Embodiment 1: A method for preparing a vesicle comprising the steps of:
a) providing recombinant Trypanosoma brucei cells expressing a VSG, preferably, a sortaggable VSG;
b) treating said cells in hypotonic solution in the presence of at least one protease inhibitor until the cells are lysed;
c) isolating the cellular membranes from the solution of step b);
d) suspending the isolated membranes obtained in step c) in an isotonic solution;
e) treating the suspended cellular membranes obtained in step d) with sonication in order to obtain a vesicle suspension;
f) removing aggregated membranous debris from the vesicle suspension obtained in step e);
g) separating the vesicle suspension into populations of vesicles; and
h) providing vesicles from a population of vesicles which is characterized by the following parameters: (i) having a single predominant protein revealed after Coomassie staining an SDS PAGE that has an apparent molecular weight of 55 to 60 kDa, (ii) having a spherical appearance in electron micrographs and (iii) exhibiting a homogenous surface structure in electron micrographs

Embodiment 2: The method of embodiment 1, wherein said population of vesicles is further characterized by an average diameter within the range of about 50 nm to about 500 nm, preferably, about 150 nm to about 250 nm, as determined by dynamic light scattering analysis.

Embodiment 3: The method of embodiment 1 or 2, wherein said recombinant Trypanosoma brucei cells lack GPI phospholipase C.

Embodiment 4: The method of any one of embodiments 1 to 3, wherein said at least one protease inhibitor is the HALT protease inhibitor composition.

Embodiment 5: The method of any one of embodiments 1 to 4, wherein said method further comprises after step c) and prior to step d) the steps of:
treating said cells in deionized water solution; and
isolating the cellular membranes from said solution.

Embodiment 6: The method of any one of embodiments 1 to 5, wherein said isotonic solution is a buffer comprising 20 mM HEPES, 150 mM NaCl₂, pH 8.

Embodiment 7: The method of any one of embodiments 1 to 6, wherein said sonication in step e) is carried for 5 minutes, 40% duty, with pulsar.

Embodiment 8: The method of any one of embodiments 1 to 7, wherein said removing aggregated membranous debris from the vesicle suspension in step f) is carried out by filtration in step f) is carried out by using a 0.45 uM filter.

Embodiment 9: The method of any one of embodiments 1 to 8, wherein said separation in step g) comprises gel filtration that is carried out through gel filtration chromatography on a 20 mM HEPES, 150 mM NaCl₂, pH 8-equilibrated Sephacryl S-500 26/60 column, a Superose 6 column or a CaptoCore column.

Embodiment 10: The method of any one of embodiments 1 to 9, wherein said method further comprises introducing a cargo agent of interest into the vesicles provided in step h).

Embodiment 11: The method of embodiment 10, wherein said introducing comprises the steps of:
a) suspending the vesicles in transfection buffer comprising an excess of cargo agent of interest;
b) carrying out electroporation; and
c) purifying loaded vesicles after electroporation.

Embodiment 12: The method of claim 10, wherein said introducing comprises the steps of:
a) freeze-drying the vesicles in the presence of the cargo;
b) resuspending the freeze-dried vesicles and cargo in a resuspension buffer;
c) mixing the resuspension mixture; and
d) purifying the loaded vesicles.

Embodiment 13: The method of any one of embodiments 10 to 12, wherein said cargo agent of interest is selected from the group consisting of: small molecule drugs, peptides, proteins, and nucleic acid molecules.

Embodiment 14: The method of any one of embodiments 1 to 13, wherein said method further comprises sortagging a targeting compound to the sortaggable VSG on the vesicles provided in step h).

Embodiment 15: The method of embodiment 14, wherein said sortagging comprises the steps of:
a) treating the vesicles by sortase in the presence of targeting compound; and
b) purifying vesicles sortagged with the targeting compound.

Embodiment 16: The method of embodiment 14 or 15, wherein said targeting compound is an antibody or nanobody recognizing a target molecule on a target cell.

Embodiment 17: A vesicle comprising a VSG, preferably, a sortaggable VSG characterized by the following parameters: (i) having a single predominant protein revealed after Coomassie staining an SDS PAGE that has an apparent molecular weight of 55 to 60 kDa, (ii) having a spherical appearance in electron micrographs and (iii) exhibiting a homogenous surface structure in electron micrographs.

Embodiment 18: The vesicle of embodiment 17, wherein said vesicle is further characterized by an average diameter within the range of about 50 nm to about 500 nm, preferably, about 150 nm to about 250 nm, as determined by dynamic light scattering analysis.

Embodiment 19: The vesicle of embodiment 17 or 18, which is loaded with a cargo agent of interest, preferably, selected from the group consisting of: small molecule drugs, peptides, proteins, and nucleic acid molecules.

Embodiment 20: The vesicle of embodiment 19, wherein said nucleic acid molecule is an antisense oligonucleotoide or an expression construct encoding it.

Embodiment 21: The vesicle of embodiment 20, wherein said antisense oligonucleotide is suitable for RNA editing, preferably, for the generation of a neoepitope in an immunogenic peptide in a cancer cell.

Embodiment 22: The vesicle of any one of embodiments 17 to 21, wherein said vesicle is sortagged with a targeting compound, preferably, an antibody or nanobody recognizing a target molecule on a target cell.

Embodiment 23: The vesicle of any one of embodiments 17 to 22, which is obtainable by the method of any one of embodiments 1 to 16.

Embodiment 24: A vesicle as defined in any one of embodiments 17 to 23 for use in treating and/or preventing a disease or medical condition.

Embodiment 25: A vesicle as defined in any one claims 17 to 23 for use as a compound delivery vehicle, preferably, a drug delivery vehicle.

Embodiment 26: A kit for carrying out the method of any one of embodiments 1 to 16 comprising recombinant Trypanosoma brucei cells expressing a sortaggable VSG and at least one agent for carrying out the method as defined in any one of embodiments 1 to 16.

Embodiment 27: The kit of embodiment 26, wherein said at least one agent is selected from the group consisting of: deionized water, at least one protease inhibitor, preferably the HALT protease inhibitor composition, a buffer comprising 20 mM HEPES, 150 mM NaCl₂, pH 8, a 0.45 uM filter, a 20 mM HEPES, 150 mM NaCl₂, and sortase.

Embodiment 28: A kit comprising the vesicle of any one of embodiments 17 to 23.

All references cited throughout this specification are herewith incorporated by reference in their entirety as well as with respect to the specifically mentioned disclosure content.

### FIGURES

FIG. 1. Schematic showing the generation and utility of the nano VAST. (A) nanoVAST are produced by sonicating osmotically lysed cell preparations of the unicellular eukaryote Trypanosoma brucei. After sonication and a series of filtration and centrifugation steps, the sonicated membranes are purified (e. g. loaded onto size exclusion columns for purification). (B) nanoVAST are cargo carrying vesicles, coated in the T. brucei surface protein VSG, that can deliver that cargo to target cells. Shown here is an example of the nanoVAST carrying a nucleic acid cargo that is then delivered to target cells; in this case B cells. (C) Since nanoVAST are coated in sortaggable VSG proteins, they can be coupled to sortaggable ligands. Shown is the linkage of nanoVAST to a nanobody specific to a B cell protein, which then facilitates an increased rate of uptake relative to (B). (D) Attaching cell-type specific ligands to the nanoVAST not only increases the delivery rate to that cell, but can also specifically facilitate delivery to that cell type among a population of additional off-target cell types.

FIG. 2. nanoVAST vesicle generation and characterization. (A-B) Gel filtration chromatograms after Superose 6 Increase resin and Sephacryl S-500 resin-based separation of sonicated T. b. brucei membrane lysate isolated from 5 billion cells' worth of material. The Y-axis indicates the total amount of protein eluting at any point throughout the runs via absorbance of light at 280 nanometers. (C) SDS-PAGE and Coomassie stain of the eluted peaks from A. VSG predominates as the major protein present in the nanoVASTs. (C) Dynamic light scattering of purified nanoVASTs reveals that they are uniform and approximately 250 nM in diameter on average.

FIG. 3. Cryo-electron micrograph of a nanoVAST vesicle. (A) Images were captured on a Talos Arctica after embedding a nanoVAST preparation on UltrAuFoil grids. This 2-dimensional representation of a nanoVAST enforces the hypothesis that the phospholipid bilayer is covered very densely with VSG protein (the hairy-looking structure surrounding the entire membrane circle) similarly to the density present on the native membrane of the live organism. Further, the scale bar (50 nM) supports the finding that the nanoVASTs are approximately 200-250 nM in diameter. (B) Images were captured on a Zeiss (Zeiss EM912 microscope at 80 kV (Carl Zeiss, Oberkochen, Germany) after nanoVAST from purified preparations were adsorbed onto glow discharged carbon coated copper grids (mesh-size 300), washed in double distilled water and negatively stained with 1% aqueous uranyl acetate. The scale bar (100 nm) supports the previous findings of the vesicles ranging from 200-250 nm.

FIG. 4. nanoVAST vesicle loading by electroporation. (A) Flow cytometry data collected from fluorescent cargo-loaded nanoVASTs after electroporation with 5 different program settings on an Amaxa Nucleofector. Program U033 is the most efficient for loading nanoVASTs. (B) Chart depicting only the U033 setting vs unstained nanoVASTs, both from (A)

FIG. 5. nanoVAST cargo delivery to target cells. Flow cytometry data collected from HEK cells incubated with cargo-loaded nanoVASTs. In (A), the nanoVASTs were loaded by electroporation with the fluorescent molecule FAM as a representative of therapeutic small molecule drug. In (B), the nanoVASTs were loaded with a fluorescently (Cy-5) labeled short RNA oligomer as a representative for e.g., CRISPR guide RNAs.

FIG. 6. VSG is detectable on the surface of nanoVAST targeted cells. Flow cytometry data collected from (A) HEK cells and (B) Ramos B cells incubated with nanoVAST. The cells were then stained with an anti-VSG antibody, revealing that the VSG protein can be found on the surface of the treated cells for at least a certain period of time post-treatment. This supports the hypothesis that nanoVAST cargo can be delivered to target cells through membrane fusion (as opposed to endocytosis, which would not deposit VSG on the cell surface). (C) Potential routes of entry and delivery by nanoVAST into cells. Fusion into cell membrane with concomitant release of cargo (i) or through endocytosis with delayed release of cargo (ii). Fused vesicles eventually are pinched (iii) and fuse to multivesicular bodies (iv). Release of cargo through endocytosis may also occur at multivesicular bodies through retrofusion (v). (D) Immunofluorescence microscopy of fixed and permeabilized nanoVAST-treated HEK cells. Multichannel composite image shows co-localization of nanoVAST with membrane-derived Rab11 endosomes adjacent to the cell nucleus. Cells were fixed, permeabilized and stained 8 hours after addition of nanoVAST particles. Nuclei and membranes were visualized with DAPI (4′, 6-Diamidino-2-phenylindole dihydrochloride) and WGA (wheat germ agglutinin Alexa Flour 594-conjugated) respectively. Recycling endosomes were stained with rabbit monoclonal anti-RAB11 (Cell Signaling Technology #5589T) and anti-rabbit IgG Alexa Fluor 488-conjugated (Invitrogen #A-11008). NanoVAST were stained with mouse monoclonal anti-VSG3 and anti-mouse IgG-Abberior® STAR RED (Sigma 52283). Images were taken using a confocal microscope, Leica TCS SP5 II.

FIG. 7. Targeting the nanoVAST to a specific cell type through sortagging facilitates delivery. (A) nanoVAST were sortagged to an anti-CD19 nanobody and incubated with Ramos B cells prior to staining with an anti-VSG antibody. The amount of VSG deposited on the target cell membrane was increased relative to non-specific nanoVAST delivery. This experiment is analogous to the schematic shown in Example 4, FIG. 6B. (B) nanoVAST sortagging was validated through FACS analysis. Flow Cytometry data of nanoVAST prepared from VSG3 expressing T. brucei that were sortagged with the fluorescent molecule TAMRA to track and measure sortagging of the vesicles.

FIG. 8. nanoVAST vesicle generation by differential centrifugation and characterization. (A) The method of isolating nano VAST from sonicated T. brucei membranes via repetitive centrifugation steps. The nanoVAST are expected to remain in the supernatant at speeds less than 20,000 g. All spins are performed at 4° C. (B) Dynamic light scattering of purified pelleted nanoVASTs compared to purified nanoVASTs from a FPLC or gel filtration column reveals that they are also uniform but smaller, approximately 150 nm in diameter on average. (C) Transmission electron microscopy imaging reveals a spherical structure with an electron-dense coating; the nanoVAST. (D) SDS-PAGE analysis by Coomassie staining the pelleted nanoVASTs reveals that VSG predominates as the major protein present in the preparation.

FIG. 9. Schematic and readout of supplementary polishing step during nano VAST delivery vesicle preparation. (A) Insertion of a polishing step using a CaptoCore resin can be at single or multiple points during preparation of the final nanoVAST delivery particle, e.g. prior to (i) or after purification (ii), after sortagging (iii) and prior to application (iv). (B) FPLC chromatograms after CaptoCore resin-based polishing of nanoVAST preparation (step ii after purification). The y-axis indicates the total amount of protein eluting at any point throughout the runs via absorbance of light at 280 nanometers. (C) Electron microscopy images were captured of nanoVAST eluted peak from (B) after adsorption onto glow discharged carbon coated copper grids (mesh-size 300), washed in double distilled water and negatively stained with 1% aqueous uranyl acetate. The vesicles remain of the appropriate size after CaptoCore purification. (D) Dynamic Light Scattering of the eluted nanoVAST peak from (B) reveals that the nanoVAST are uniform and approximately 150 nm in diameter on average. (E) SDS-PAGE and Coomassie staining of the eluted peak from (B). VSG predominates as the major protein present in the nanoVAST.

FIG. 10. Freeze-drying as an alternative method of loading nanoVAST vesicle (A) To load RNA cargo into nanoVAST using freeze drying method, 9 μg VSG protein worth of nanoVAST was mixed with 42 pmol cy5-tagged RNA in RNAse-free centrifuge tubes. The amount of nanoVAST used was determined by SDS page quantification with reference to a VSG3 protein standard. The samples were loaded into the freeze drier (Alpha 1-2 LSCbasic-Martin Christ) and the drying time required is dependent on the volume of the sample. The freeze-dried samples were then rehydrated with HEPES buffer (20 mM HEPES, 150 mM NaCl) and mixed by pipetting up and down, and vortexing gently. After which, the samples were centrifuged at 20,000 g at 4° C. for 30 minutes to remove any unloaded RNA cargo. The resulting nanoVAST pellet was resuspended in 1×PBS to assess RNA loading and for HEK cell feeding experiments. (B) Dynamic Light Scattering graph showing size distribution of the freeze dried-rehydrated nanoVAST vesicles. The vesicles are uniform and have an average diameter of approximately 200 nm. (C) Transmission electron microscopy image of a nanoVAST vesicle. The image confirms that the structure of nanoVAST is preserved after freeze drying. (D) SDS PAGE analysis of the freeze dried-hydrated nanoVAST showing VSG3 as the dominant protein. (E) Western blot validating that nanoVAST surface protein VSG3 is preserved after the freeze drying process. (F) Flow cytometry data collected from freeze dried-hydrated nanoVASTs loaded with Cy-5 tagged RNA by freeze drying followed by rehydration. This results in efficient RNA loading.

FIG. 11. Cargo uptake by HEK cells through electroporation and freeze-drying. (A) HEK cells fed with nanoVAST that were loaded with Cy5-tagged RNA by electroporation. The chart shows HEK cells that have taken up nanoVAST as indicated with a positive VSG3 signal. (i) VSG3 negative (ii) and VSG3 positive (iii) were selected and assessed for presence of Cy5 fluorescence, as an indicator of RNA uptake. VSG3 positive cells showed remarkable uptake of Cy5-tagged RNA (iii). (B) In comparison, HEK cells fed with nanoVAST that were loaded with Cy5-tagged RNA by freeze-drying as described in FIG. 10A. The chart shows HEK cells that have taken up nanoVAST as indicated with a positive VSG3 signal (i). VSG3 negative (ii) and VSG3 positive (iii) were selected and assessed for presence of Cy5 fluorescence, as an indicator of RNA uptake. VSG3 positive cells showed remarkable uptake of Cy5-tagged RNA (iii).

FIG. 12. nanoVAST typing can be altered through VSG modification or selection. nanoVAST type can be altered through: (i) use of sortagging of peptides, e.g., peptide ligands, antibodies, or nanobodies; (ii) use of VSG gene modification through addition of encoded targeting peptide tag, e.g., listed in Table 1; and (iii) complete VSG switch through selection from approximately 2000 different VSG genes encoded in the T. brucei genome. Some examples of VSG proteins (top view) of the exposed region as a molecular surface shaded according to polarity (white to grey: polar to hydrophobic). Differences in surface topography, polarity, or other physical properties, may affect nanoVAST cell tropism or physical properties.

FIG. 13. Purification of nanoVAST from T.brucei expressing Iltat (ILTat1.24) protein as an alternative to VSG3. (A) Dynamic Light Scattering of Iltat nanoVAST purified with differential centrifuging method described in FIG. 8 reveals that they are uniform and approximately 150 nm in diameter on average similar to VSG3 nanoVAST. (B) Electron microscopy images were captured of ILtat nanoVAST after adsorption onto glow discharged carbon coated copper grids (mesh-size 300), washed in double distilled water and negatively stained with 1% aqueous uranyl acetate. The scale bar (250 nm) supports the previous findings of the vesicles ranging from 200-250 nm. (C) SDS-PAGE and Coomassie stain of the Iltat nanoVAST. Iltat predominates as the major protein present in the nanoVAST which illustrates the successful purification of different nanoVAST types is possible. (D) nanoVAST sortagging was validated through FACS analysis. Flow Cytometry data of nanoVAST prepared from ILtat expressing T.brucei that were sortagged with the fluorescent molecule TAMRA to track and measure sortagging of the vesicles.

FIG. 14. RAW 264.7 macrophages do not produce TNF-α in response to stimulation by nanoVAST. Cells were stimulated with inactivated E. coli, 100 nM Monophosphoryl lipid A (MPLA), and 10 nM nanoVAST. Media was collected from wells at the specified times for ELISA analysis. Each data point represents the mean±SD of triplicate wells. The Y axis depicts ELISA signal by raw absorbance from the plate reader.

### EXAMPLES

The Examples merely illustrate the invention. They shall not, whatsoever, construed as limiting the scope of the invention.

### Example 1: Nano VAST Vesicle Preparation

Approximately 1-10 billion T. brucei expressing sortaggable VSG and lacking the GPI-phospholipase C gene were grown in standard HMI-9 (with 10% fetal bovine serum) and were isolated by centrifugation (2,000 g for 20 minutes at room temperature).

Cells were washed once with phosphate buffered saline (PBS) to remove residual extracellular proteins, and then the resulting pellet was lysed for 10 minutes on ice through suspension in 5 mL of ice-cold diH₂O with HALT protease inhibitors for lysis.

The lysed suspension was the centrifuged (10,000 g for 10 minutes at 4° C.) to isolate the membranous material and remove the cytoplasmic contents. The ice-cold diH₂O extraction and centrifugation process was repeated twice.

The membrane pellet was then suspended in 2 mL of 20 mM HEPES, 150 mM NaCl₂, pH 8 and sonicated (5 minutes, 40% duty, with pulsar) on ice to sheer the large membranes into relatively small vesicles. The suspension was then centrifuged (2,000 g for 5 minutes at 4° C.) to remove any remaining large aggregated pelletable debris (for example, cells that remained intact) and filtered through a 0.45 uM filter using a 2.5 mL syringe. The filtered supernatant was then sonicated and centrifuged again with the same process and the new supernatant was filtered once more to result in a finer solution, free from aggregated cell debris and suitable for gel filtration.

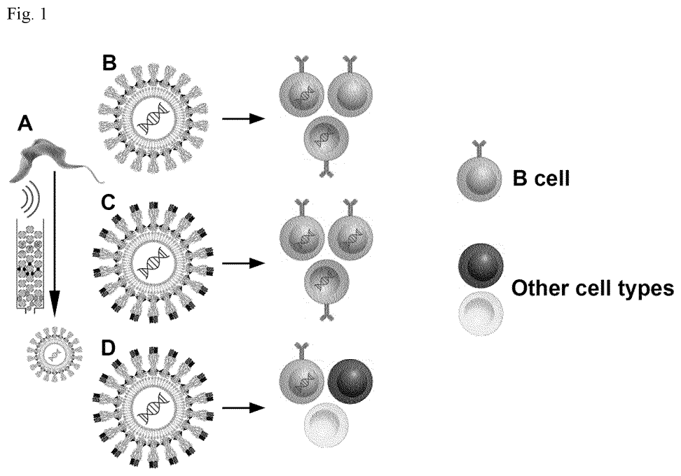

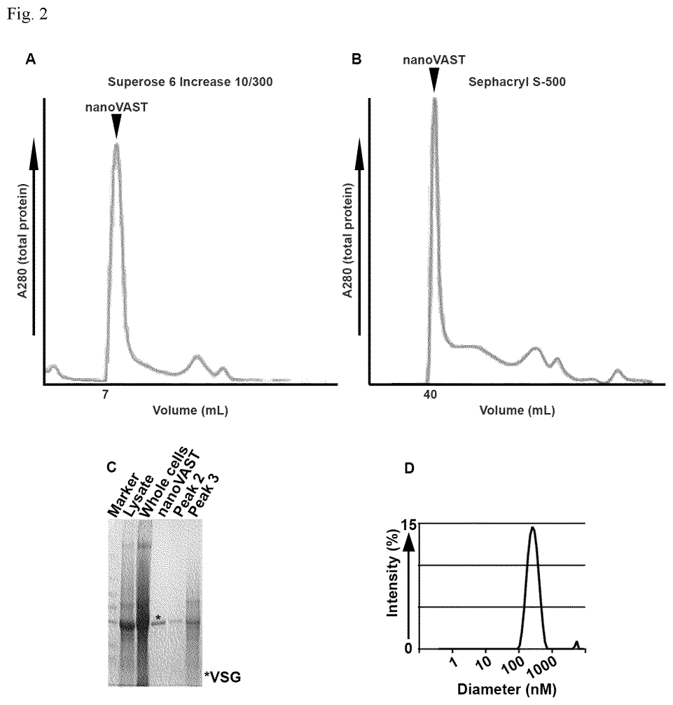

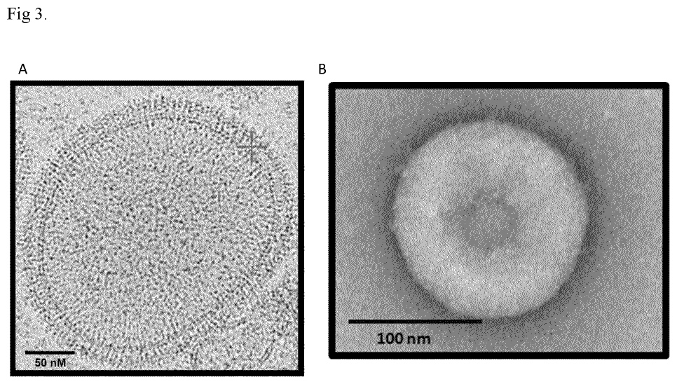

The filtered vesicle suspension was then separated by gel filtration chromatography on a 20 mM HEPES, 150 mM NaCl₂, pH 8-equilibrated Sephacryl S-500 26/60 column (FIG. 1A, FIG. 2A) in order to remove small debris and isolate the largest vesicles, the latter of which account for the majority of the contents of the preparation and are henceforth referred to as “nanoVASTs”.

The nanoVASTs were then subjected to a variety of characterizations, including SDS-PAGE for protein purity and dynamic light scattering for vesicle size and uniformity determinations (FIGS. 1B and C, FIG. 2). Specifically for dynamic light scattering, a ZetaSizer Nano (Malvern Playtical) was used for the measurement and the accompanying Zetasizer software was used for the analysis of the vesicles. 20 ul of the sample were diluted in 1 ml of PBS and placed into disposable polystyrene cuvettes and then measured.

The following parameters were found to be characteristic for nanoVAST vesicles:
(i) having a single predominant protein revealed after Coomassie staining an SDS PAGE that has an apparent molecular weight of 55 to 60 kDa,
(ii) having a spherical appearance in electron micrographs and
(iii) exhibiting a homogenous surface structure in electron micrographs

Moreover, the vesicles, typically, had an average diameter of 250 nm identified by dynamic light scattering,

### Example 2: Nano VAST Loading

To then load the nanoVAST with cargo, 0.5 mg of protein worth of nanoVASTs (determined by the colorimetric BCA protein quantification assay and by SDS page comparative quantification to a protein standard) were suspended in homemade transfection buffer (90 mM Na₂HPO₄, pH 7.3, 5 mM KCl, 0.15 mM CaCl₂), 50 mM HEPES, pH 7.3) and electroporated in the presence of a molar excess of the cargo of interest (the concentration of the cargo is molecule dependent).

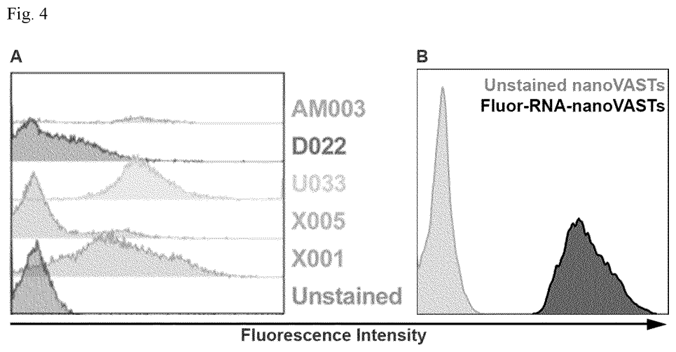

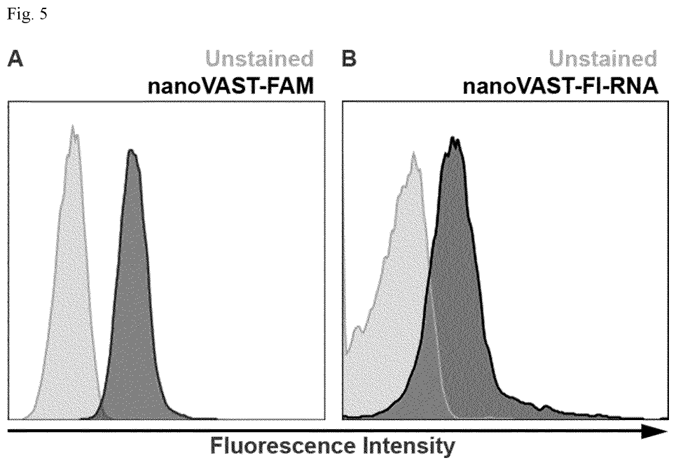

Electroporation was conducted using an Amaxa Nucleofector 2b with the U033 program after assessing nanoVAST transfection efficiency using a wide array of program options (e.g., FIG. 4). Cargo-loaded nanoVASTs were isolated from free-cargo by centrifugation (20,000 g, 4° C., 30 min) and washed multiple times.

The produced nanoVAST were capable of being detected with flow cytometry methods. In the case that the cargo of choice is fluorescently labeled, both cargo-loaded and cargo-free nanoVAST could be measured for their fluorescence intensity through flow cytometry (shown in FIG. 4). This facilitated the determination of loading efficiency. With the use of the U033 program for electroporation there was a >99% efficiency of nanoVAST loading (FIG. 4A). For the described experimental setup, a BD FACSCalibur™ was used.

### Example 3: Nano VAST Delivery

Cells treated with cargo-loaded nanoVASTs will incorporate them, thereby taking up the molecule of interest. These molecules can be hypothetically anything small enough to fit into the nanoVASTs, including small molecule drugs (represented by the fluorescent molecule FAM—FIG. 5A) and nucleic acids (represented by a fluorescent RNA molecule—FIG. 5B).

For the treatment, cells were plated in 24 well plates in appropriate densities. A mixture of fluorescent cargo-filled nanoVAST and appropriate cell media was prepared. The amount of nanoVAST that was used could vary between 100 to 5000:1 ratio of vesicles to cells depending on the setup.

The aforementioned mixture of nanoVAST and media was then added drop by drop to each cell culture well and cells were incubated in an incubator (37° C., 5% CO₂) for at least 1.5 hours before further analysis.

Cells were collected and washed with PBS three times (100 g for 7 minutes in room temperature). After flow cytometry, it was determined that treated cells had an increase in their fluorescence which suggests they incorporated the cargo delivered by nanoVAST (FIG. 5).

### Example 4: Nano VAST Delivery Facilitated by Membrane Fusion

The predominant protein of the nanoVASTs (VSG3) was present on the surface of both cell lines tested (Ramos B-cells and HEK 293T cells) after nanoVAST treatments. This suggests membrane fusion as the mechanism of delivery.

The inventors determined this through the following experimental setup. As described in example 3, cells were plated in 24 well plates in appropriate densities for each cell line. A mixture of nanoVAST and appropriate cell media (RPMI 1640 for the Ramos B-cell line and DMEM for HEK 293T cells, both supplemented with 10% FBS) was prepared. It was added drop by drop to each well and then cells were incubated for at least 1.5 hours.

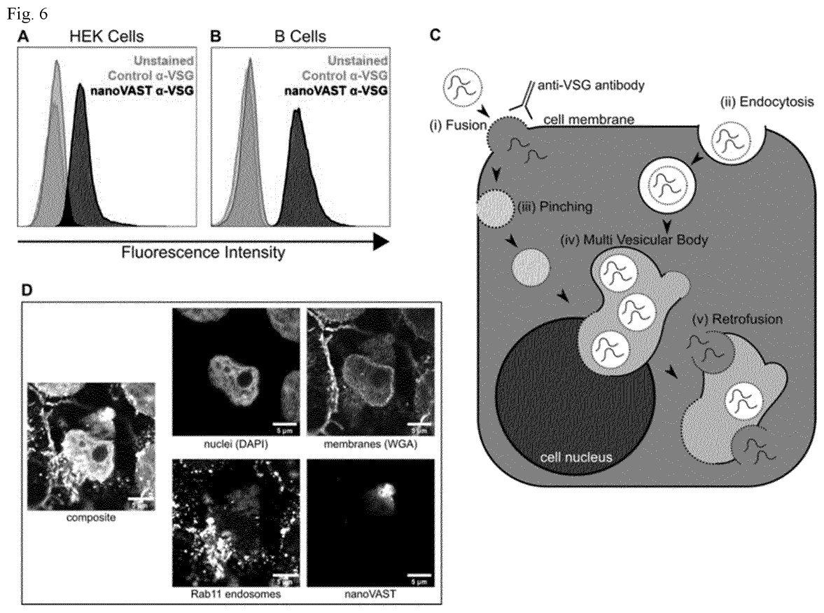

Cells were collected and washed with PBS three times (100 g for 7 minutes in room temperature) and then were stained with an anti-VSG3-FITC conjugated antibody for 10 minutes on ice. After washing with PBS 3 times (100 g for 7 minutes in room temperature), the flow cytometry results showed a clear increase in VSG3 presence on the surface of the cells (FIG. 6A-B). Based on the fact that normally, VSG3 is completely absent from the surface of these cell lines, the conclusion was reached that its presence was a direct result of the interaction with the VSG3 coated nanoVASTs through membrane fusion. If nanoVAST had been imported instead through endocytosis, there would not have been any surface-detectable VSG as it too would have been internalized along with the cargo.

FIG. 6C illustrates the cellular mechanisms of nanoVAST cargo delivery into cells, either through membrane fusion and direct release of cargo (FIG. 6C, i) or through endocytosis (FIG. 6C, ii) and later release of the cargo (FIG. 6C, v). Either delivery mechanism leads to a release of cargo into the cell, and the internalization and accumulation of VSG3 into endocytic vesicles (FIG. 6C, iv). The internalized VSG3 could be detected 8 hours post-nanoVAST treatment of the cells, in this case HEK cells, in RAB11-positive endocytic vesicles (FIG. 6D).

### Example 5: Nano VAST Targeted Delivery

To then sortag the nanoVASTs with a targeting component of interest (e.g., an antibody or nanobody that binds to a specific target cell surface marker), the loaded nanoVASTs were then incubated at 37° C. for 3 hours with gentle agitation in the presence of purified recombinant Streptococcus pyogenes Sortase A (100 uM), 30 mM CaCl₂), and the sortaggable molecule/targeting component of interest (300 uM).

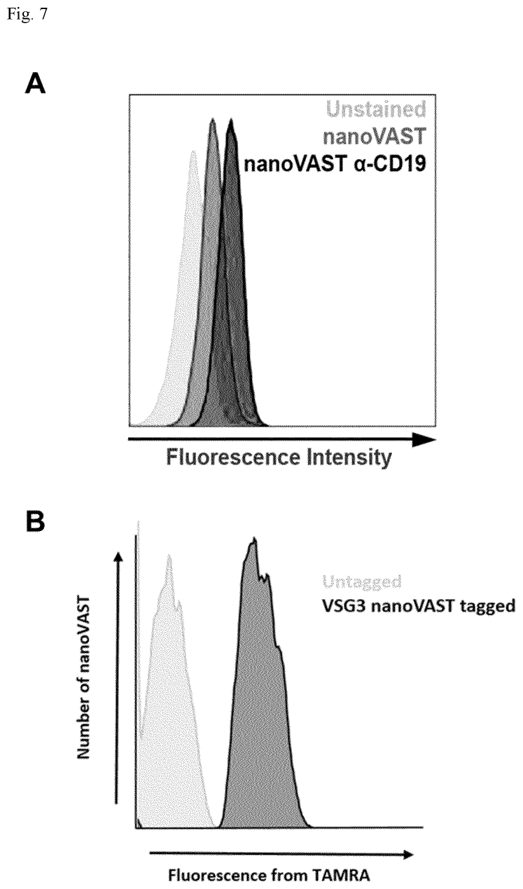

The sortagged nanoVASTs were then re-isolated from the free sortagging reagents by centrifugation (20,000 g, 4° C., 30 min) and stored in 20 mM HEPES, 150 mM NaCl₂, pH 8. nanoVAST sortaggability was assessed using a sortaggable fluorescent molecule, TAMRA. The vesicles were sortagged with the aforementioned protocol and then analyzed with flow cytometry. FIG. 7B illustrates that nanoVAST can be efficiently sortagged. The inventors proceeded to then sortag a functionally relevant targeting moiety.

In this example, the targeting molecule sortagged under the aforementioned conditions was an antiCD19 nanobody which would target the CD19 receptors on the surface of Ramos B-cells. Ramos B-cells were treated with sortagged nanoVASTs as explained in example 3 and an antiVSG3 staining of the cells followed, as described in example 4. As shown in FIG. 7A, B-cells treated with the targeted nanoVASTs incorporated more VSG3 in their membranes than the respective ones treated with unspecific nanoVAST. This implies that the invention can be targeted, resulting in increased efficiencies of delivery.

### Example 6: nanoVAST Vesicle Preparation with Alternative Method

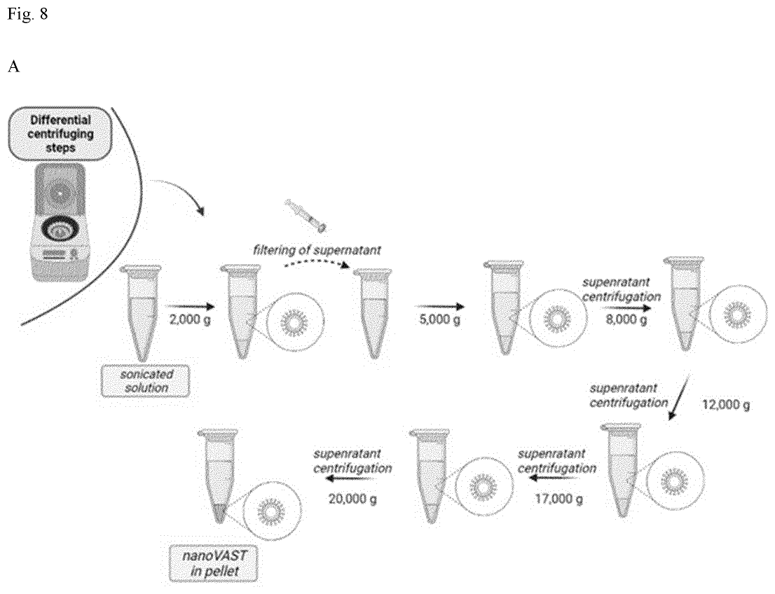

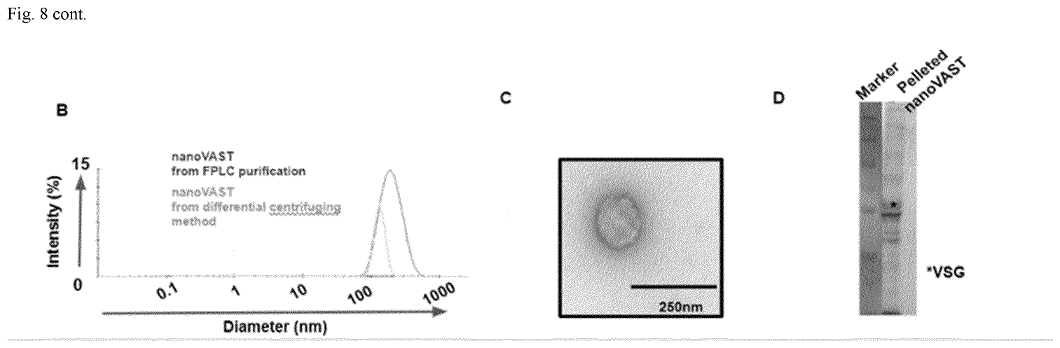

As an alternative method, the filtered supernatant from step f of the first embodiment of the present invention may be subjected to several centrifugation steps to gradually eliminate more cellular and membrane debris. It was observed that nanoVASTs can be pelleted at speeds equal or greater than 20,000 g. Therefore, by gradually increasing the speed of repeated centrifugations (FIG. 8A), it is possible to progressively pellet aggregated membranous debris prior to pelleting the nanoVAST while leaving soluble proteins in the final supernatant. 5 different speeds were used, ranging from 5,000 to 20,000 g. Supernatant after each spin is collected and moved to a clean tube. The spin at the chosen speed is repeated until pellets are no longer formed, indicating that relatively heavy debris that could be eliminated at this speed has indeed been removed. This typically occurs after 2 to 5 rounds. Once the pellet is removed, the supernatant is centrifuged at a higher speed. All the spins lower than 20,000 g are performed at 4° C. for 5 min. The resulting supernatant after 17,000 g centrifugation is transferred to a new tube and centrifuged at 20,000 g for 30 min at 4° C. At this stage, the nanoVASTs are found in the pellet. The pellet is resuspended in 20 mM HEPES, 150 mM NaCl₂, pH 8.0 and centrifuged again at 20,000 g for 30 min at 4° C. as an additional cleaning step. The pellet now contains nanoVASTs and can be stored.

The nanoVASTs resulting from this alternative method, were examined by Dynamic Light Scattering, by Transmission Electron Microscopy and by SDS PAGE (FIG. 8B-D) as previously described, which indicated that this method can be used to purify a nanoVAST population. The nanoVASTs purified by differential centrifugation were a more uniform population of approximately 150 nM in diameter, but were otherwise similar to those purified by gel filtration/FPLC.

For said experiments, approximately 1-10 billion T. brucei expressing sortaggable VSG and lacking the GPI-phospholipase C gene were grown in standard HMI-9 (with 10% fetal bovine serum) and were isolated by centrifugation (2,000 g for 20 minutes at room temperature). Cells were washed once with phosphate buffered saline (PBS) to remove residual extracellular proteins, and then the resulting pellet was lysed for 10 minutes on ice through suspension in 5 mL of ice-cold diH₂0 with HALT protease inhibitors for lysis. The lysed suspension was then centrifuged (10,000 g for 10 minutes at 4° C.) to isolate the membranous material and remove the cytoplasmic contents. The ice-cold diH₂O extraction and centrifugation process was repeated twice. The membrane pellet was then suspended in 2 mL of 20 mM HEPES, 150 mM NaCl₂, pH 8 and sonicated (5 minutes, 40% duty, with pulsar) on ice to sheer the large membranes into relatively small vesicles. The suspension was then centrifuged (2,000 g for 5 minutes at 4° C.) to remove any remaining large aggregated pelletable debris (for example, cells that remained intact) and filtered through a 0.45 uM filter using a 2.5 mL syringe. The filtered vesicle suspension was then subjected to a differential centrifugation protocol as shown in FIG. 8A.

A 5,000 g centrifuging step at 4° C. for 5 min was repeated 3 times; until no pellet was present. The resulting supernatant was centrifuged at 8,000 g at 4° C. for 5 min, a step which was repeated 2 times; until no pellet was present. Similarly, the supernatant from this step was centrifuged at 12,000 g at 4° C. for 5 min. This step was repeated 2 times; until no pellet was present. The supernatant was then centrifuged at 17,000 g at 4° C. for 5 min. After 2 spins, the supernatant was moved to a new tube and was centrifuged at 20,000 g at 4° C. for 30 min. An additional washing step followed, where the pellet was resuspended in 20 mM HEPES, 150 mM NaCl₂, pH 8 and re-centrifuging at 20,000 g at 4° C. for 30 min. The resulting pellet was examined by Dynamic Light Scattering, by transmission electron microscopy, and by SDS PAGE (FIG. 8B-D), revealing that spherical VSG-coated vesicles were isolated.

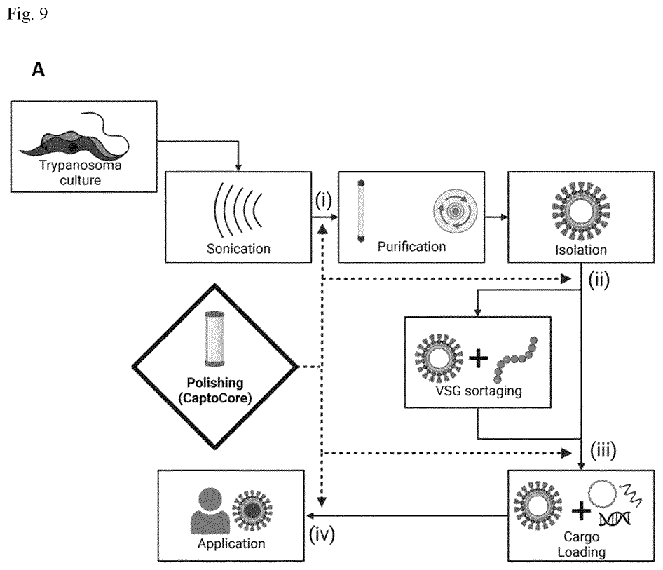

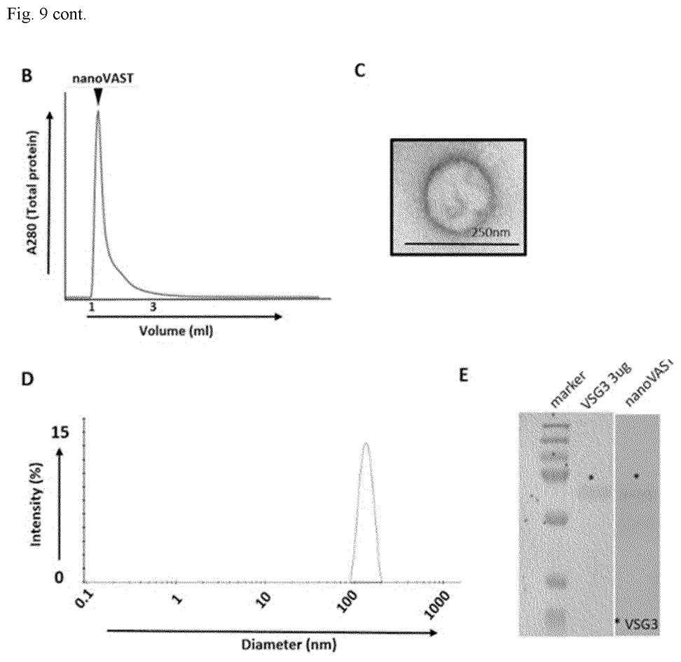

Regardless of which purification methodology is deployed, nanoVAST preparations can also then be polished using CaptoCore resin. Polishing is done through running a crude or semi-processed nanoVAST preparation through a CaptoCore 700 (Cytiva) resin column. It could be inserted as a supplementary step at one or several steps during nanoVAST preparation, loading or “sortagging” as needed (FIG. 9A). It separates the larger nanoVAST vesicles and retains other particles smaller than the 700 kDa molecular weight cutoff; thus, it removes nucleic acids, non-nanoVAST proteins, and other cellular debris.

The inventors then investigated whether nanoVAST can also be loaded with alternative strategies to electroporation, which may be beneficial/required for certain applications. Here freeze-drying is described as a novel alternative method of loading RNA cargo into nanoVAST vesicles.

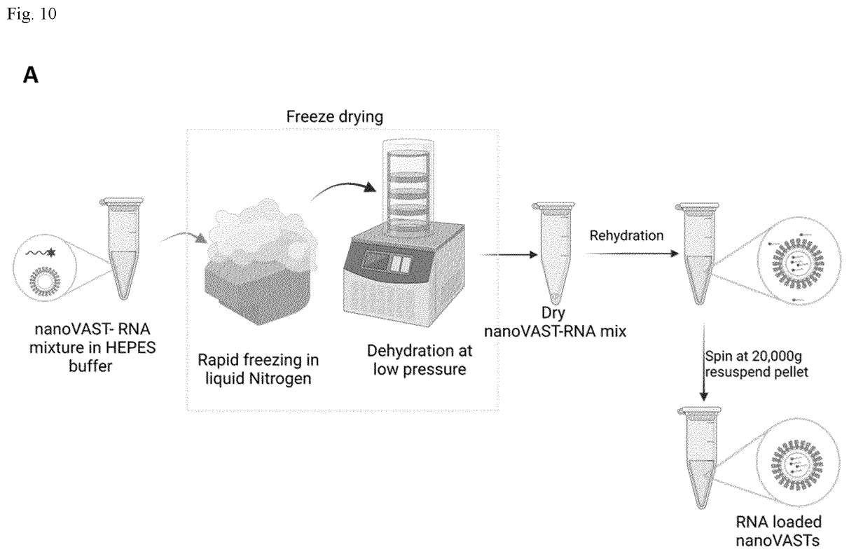

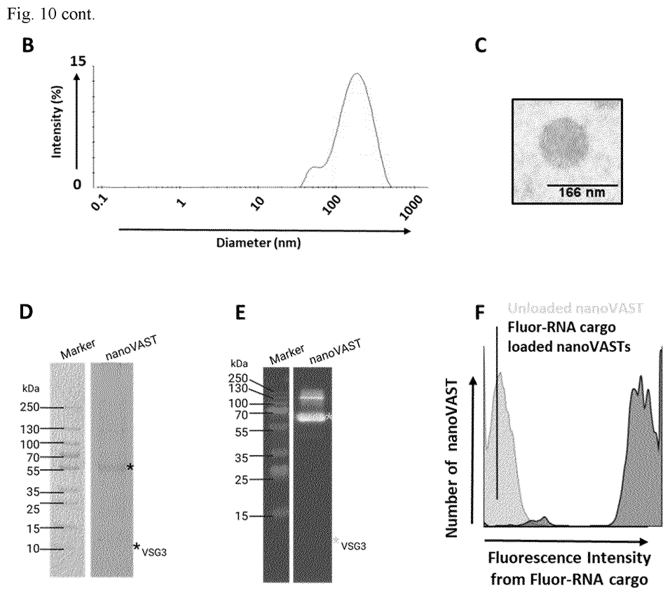

The freeze-drying loading process (FIG. 10A) involved mixing nanoVASTs and fluorescently-labeled RNA in HEPES buffer (20 mM HEPES, 150 mM NaCl). The nanoVAST-RNA mix was then rapidly frozen in liquid nitrogen. After which the frozen sample was quickly transferred to a freeze-drier (Alpha 1-2 LSCbasic-Martin Christ), where the samples were dehydrated at low pressure. The resulting product was a dried nanoVAST-RNA formulation.

After freeze-drying, the quality of the nanoVASTs was studied by checking presence and quality of the VSG3 protein and assessing the size and structure of the nanoVASTs. To do this, dried nanoVASTs that had not been co-dried with the RNA cargo were hydrated in HEPES buffer. After that, SDS PAGE analysis was performed and the result showed that VSG3 protein was still intact after freeze-drying (FIG. 10D). The presence of VSG3 was further confirmed by western blotting (FIG. 10E). The size and uniformity of the vesicles were determined by dynamic light scattering and this was done as described in Example 1. As illustrated in FIG. 10B, the expected size and uniformity were maintained after freeze-drying. Finally, re-hydrated nanoVAST vesicles were analyzed by transmission electron microscopy and the vesicles' standard round appearance remained intact (FIG. 10C). Taken together, these analyses confirmed freeze-drying does not alter the structure and quality of the nanoVASTs. Hence, freeze-dried nanoVASTs can be used for loading and delivery of cargo.

In this example, freeze-dried nanoVASTs were loaded with fluorescently-labeled RNA. The dried nanoVAST-RNA formulation was re-hydrated with HEPES buffer, resulting in the uptake of RNA. nanoVASTs loaded with RNA were isolated from unloaded RNA by centrifuging at 20,000 g for 30 min at 4° C. Since the RNA cargo was labeled with a fluorescent tag, loaded nanoVASTs could be detected by flow cytometry, where RNA loaded nanoVASTs were differentiated from unloaded nanoVASTs by assessing the intensity of fluorescence. As illustrated in FIG. 10F, nanoVASTs were efficiently loaded with RNA (FIG. 10F) and loading capacity was comparable to that of electroporation (FIG. 4B). This result demonstrates freeze-drying as an alternative method of loading nanoVASTs. The advantage of having an additional loading method to electroporation is that each of the approaches may be suited for loading specific cargo types. Therefore, this invention expands the capability of the nanoVAST loading platform.

### Example 7: Nano VAST Vesicle Loading with an Alternative Method

Having demonstrated that freeze-dried-hydrated nanoVASTs can be loaded with RNA cargo (FIG. 10F), it was evaluated if the vesicles could deliver RNA into HEK 293T cells. The appropriate density of HEK cells was plated in a 24 well plate as described in Example 3. After which, RNA-loaded freeze-dried nanoVASTs in DMEM cell media were slowly added, drop by drop to each cell culture well, and then incubated for at least 1.5 hours in a 37 C, 5% CO₂ humidified environment.

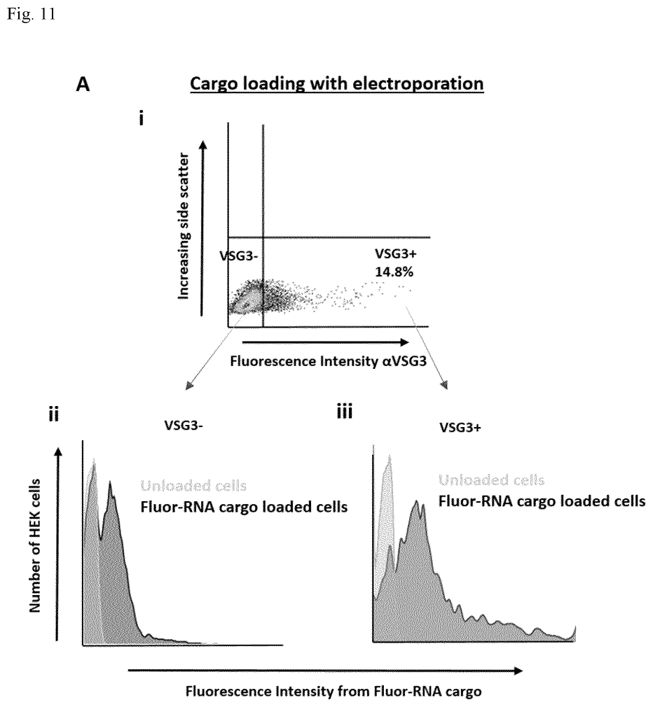

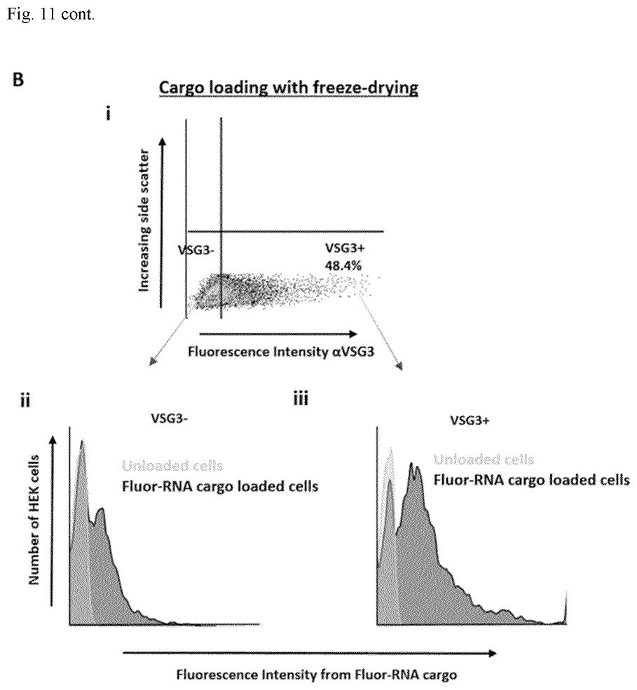

After this incubation, the cells were harvested and washed 2 times with 1×PBS by centrifuging at 100 g for 5 minutes at room temperature. To identify HEK cells that had taken up nanoVASTs, the washed cells were incubated on ice for 30 minutes with 1:100 FITC conjugated anti VSG3 antibody. The samples were analyzed by flow cytometry to identify cells that had taken up nanoVASTs and RNA cargo. HEK cells that took up nanoVASTs were VSG3 positive and this accounted for 48.4% of the HEK cell population (FIG. 11B.i). This population was also loaded with RNA as depicted by the increased intensity of the fluorescence tagged RNA (FIG. 11B.iii). There was also a proportion of HEK cells with no VSG3 signal but loaded with the RNA cargo (FIG. 11B.ii). This finding suggested that there is a proportion of nanoVASTs that lose the VSG3 surface protein upon uptake but still release RNA cargo into the HEK cells. The results from the freeze-dried nanoVAST HEK feeding experiments were comparable to those of the electroporated nanoVAST-fed HEK cells (FIG. 11A). From this data it was concluded that freeze-dried nanoVASTs can deliver cargo into target cells. Again here, highlighting that this novel method of delivering cargo expands the application of the inventors' nanoVAST loading platform.

### Example 8: Preparation of Nano VAST from Different VSG-Expressing Trypanosomes

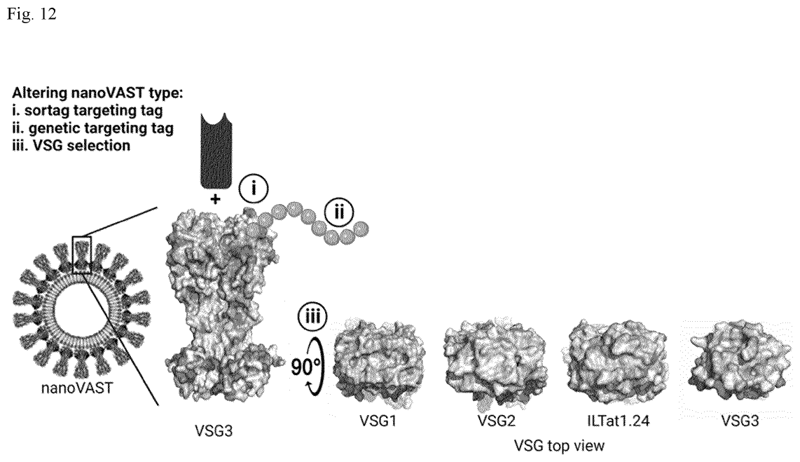

As described earlier in Example 5, nanoVAST cell specificity or targeting can be achieved through alteration of the VSG coat of the nanoVAST. In the case of Example 5, an anti-CD19 targeting nanobody, a “targeting moiety”, was covalently bound to the VSG3-coat of the nanoVAST with sortase. However, several additional methods could be used to alter the VSG coat to switch nanoVAST targeting or cell specificity, e.g., addition of different “targeting moieties” or a change of the physicochemical properties of the VSG (FIG. 12). Besides direct nanoVAST “sortagging”, one can genetically modify VSG genes of trypanosomes, from which nanoVAST would be made, to directly encode peptide-based “targeting moieties” (FIG. 12ii). Such examples of peptide-based “targeting moieties”, though not limited to these, are listed in Table 1 which can direct nanoVAST vesicles to specific cells based on design. Additionally, one could completely switch the trypanosome VSG coat to another genomically encoded VSG gene that translates to VSGs with completely different electrochemical properties that have different cell affinities, thus affecting nanoVAST cell targeting (FIG. 12iii).

TABLE 1
nanoVAST VSG targeting peptide tag examples.

| Peptide | | Length | Biological | Molecular | |
|---|---|---|---|---|---|
| Name | Peptide Sequence | (aa) | Target | Target | Paper DOI |
| GE11 | YHWYGYTPQNVI | 12 | BrCa and | EGFR | 10.1038/mt.2012.180 |
| | (SEQ ID NO: 1) | | other high | (receptor) | |
| | | | EGFR cancers | | |
| TfR- | THRPPMWSPVWP | 12 | glioma | transferrin | DOI: 10.1038/s41598- |
| T12 | (SEQ ID NO: 2) | | | receptor | 017-03805-7 |
| 15K | IRKAHCNISRAKWND | 15 | HIV-1 | chemokine | doi:10.1371/journal.p |
| | (SEQ ID NO: 3) | | receptor on | receptors | one.0014474 |
| | | | T cells and | | |
| | | | dendritic | | |
| | | | cells | | |
| 15D | IRKAHCNISRADWND | 15 | HIV-1 | chemokine | doi:10.1371/journal.p |
| | (SEQ ID NO: 4) | | receptor on | receptors | one.0014474 |
| | | | T cells and | | |
| | | | dendritic | | |
| | | | cells | | |

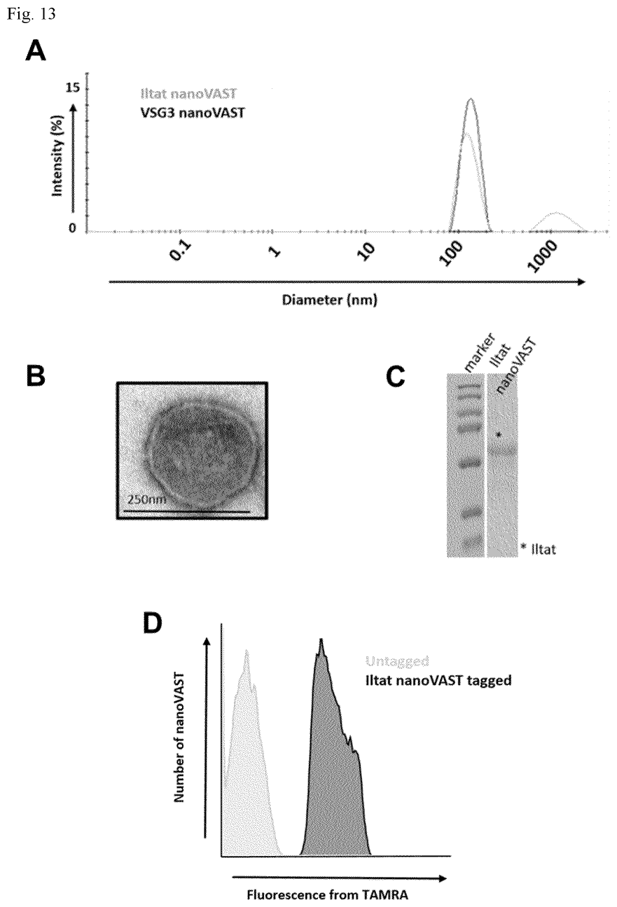

The inventors have already managed to produce nanoVASTs form trypanosomes expressing a different VSG to VSG3, namely ILtat 1.24 (so called ILtat). Vesicles prepared from ILtat-expressing trypanosomes showed similar physical properties to their VSG3 counterparts (FIG. 13A-C). Their size and integrity as well as their purity were very similar to VSG3 nanoVASTs. Additionally, ILtat nanoVASTs could be sortagged with the same sortaggable fluorescent molecule, TAMRA, that was successful with VSG3 nanoVASTs (described in Example 5, FIG. 7B). With the sortagging method described in Example 5 and Flow cytometry for validation, it was shown that ILtat vesicles can be as efficiently sortagged, thus maintaining their functional protein layer.

### Example 9: Nano VAST Immunogenicity

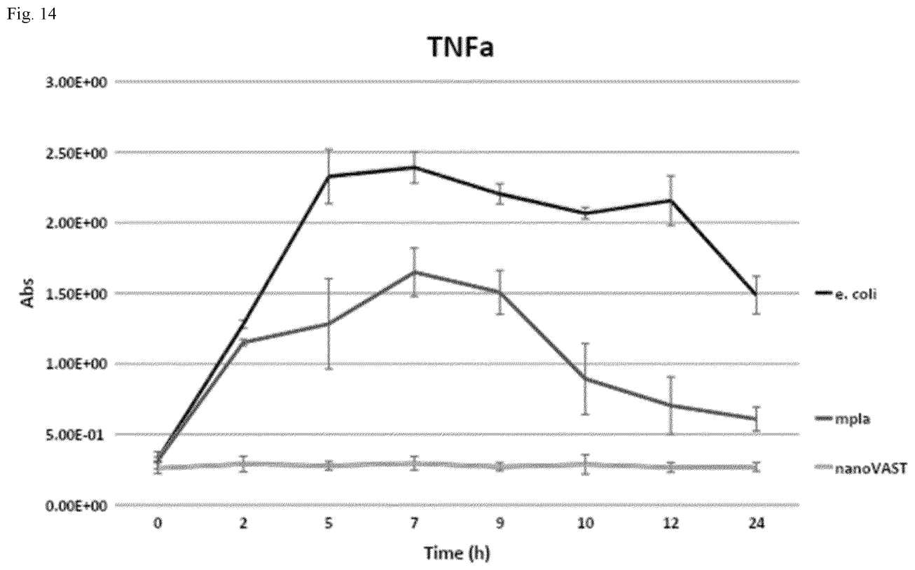

nanoVAST is an in vivo delivery tool for the specific delivery of cargos to certain cell types. All in vivo delivery tools must be analyzed for their immunogenicity to confirm that the treatment itself will not set off several different inflammatory signaling pathways and thus, lead to toxicity. The most relevant cell types to investigate would be macrophages, as they are the sentinels of the immune system and often coordinate these cytokine signaling pathways. Here, the inventors have assessed immunogenicity using an in vitro macrophage system based on the RAW cells. nanoVAST was unable to stimulate any cytokine production in these cells, compared to inactivated E. coli or MPLA, which are well-understood positive controls for such an experiment (FIG. 14).

### CITED LITERATURE

Kuzmin, D. A., Shutova, M. V., Johnston, N. R., Smith, O. P., Fedorin, V. V., Kukushkin, Y. S., van der Loo, J. C. M., and Johnstone, E. C. (2021). The clinical landscape for AAV gene therapies. Nat Rev Drug Discov 20, 173-174
Kruzik, A., Fetahagic, D., Hartlieb, B., Dorn, S., Koppensteiner, H., Horling, F. M., Scheiflinger, F., Reipert, B. M., and de la Rosa, M. (2019). Prevalence of Anti-Adeno-Associated Virus Immune Responses in International Cohorts of Healthy Donors. Mol Ther Methods Clin Dev 14, 126-133.
Wu, Z., Yang, H., and Colosi, P. (2010). Effect of genome size on AAV vector packaging. Mol Ther 18, 80-86.
Miesbach, W., Meijer, K., Coppens, M., Kampmann, P., Klamroth, R., Schutgens, R., Tangelder, M., Castaman, G., Schwable, J., Bonig, H., et al. (2018). Gene therapy with adeno-associated virus vector 5-human factor IX in adults with hemophilia B. Blood 131, 1022-1031.
Gao, K., Li, M., Zhong, L., Su, Q., Li, J., Li, S., He, R., Zhang, Y., Hendricks, G., Wang, J., et al. (2014). Empty Virions In AAV8 Vector Preparations Reduce Transduction Efficiency And May Cause Total Viral Particle Dose-Limiting Side-Effects. Mol Ther Methods Clin Dev I, 20139.
Lokugamage, M. P., Sago, C. D., and Dahlman, J. E. (2018). Testing thousands of nanoparticles in vivo using DNA barcodes. Curr Opin Biomed Eng 7, 1-8.
Cross 2014, Mol. Biochem. Parasitol. 195 (1): 59-73;
Dramsi 2005, Res. Microbiol. 156 (3): 289-97,
Comfort 2004, Infect Immun., 72 (5): 2710-22,
Chen 2011, Proc Natl Acad. Sci. USA. July 12; 108 (28): 11399
Pallen 2001, TRENDS in Microbiology, 2001, 9 (3), 97-101
WO2010/087994,
WO2011/133704,
WO2020/84072
Pinger 2017, Nat Commun. 8 (1): 828
Bobis-Wozowicz, S., Kmiotek, K., Sekula, M., Kedracka-Krok, S., Kamycka, E., Adamiak, M., Jankowska, U., Madetko-Talowska, A., Sarna, M., Bik-Multanowski, M., et al. (2015). Human Induced Pluripotent Stem Cell-Derived Microvesicles Transmit RNAs and Proteins to Recipient Mature Heart Cells Modulating Cell Fate and Behavior. Stem Cells 33, 2748-2761.
Almeida, B., Nag, O. K., Rogers, K. E., and Delehanty, J. B. (2020). Recent Progress in Bioconjugation Strategies for Liposome-Mediated Drug Delivery. Molecules 25.
WO2021/214043
Ohno S., Takanashi M., Sudo K. et al. (2012). Systemically injected exosomes targeted to EGFR deliver antitumor microRNA to breast cancer cells. Mol Ther. 2013 January; 21 (1): 185-91
Mu, L M., Bu, Y Z., Liu, L. et al. (2017). Lipid vesicles containing transferrin receptor binding peptide TfR-T12 and octa-arginine conjugate stearyl-Rs efficiently treat brain glioma along with glioma stem cells. Sci Rep 7, 3487
Chertov O, Zhang N, Chen X, Oppenheim J J, Lubkowski J, McGrath C, et al. (2011) Novel Peptides Based on HIV-1 gp120 Sequence with Homology to Chemokines Inhibit HIV Infection in Cell Culture. PLOS ONE 6 (1): e14474

1. A method for preparing a vesicle comprising the steps of:
a) providing recombinant Trypanosoma brucei cells expressing a VSG, preferably, a sortaggable VSG;
b) treating said cells in hypotonic solution in the presence of at least one protease inhibitor until the cells are lysed;
c) isolating the cellular membranes from the solution of step b);
d) suspending the isolated membranes obtained in step c) in a isotonic solution;
e) treating the suspended cellular membranes obtained in step d) with sonication in order to obtain a vesicle suspension;
f) removing aggregated membranous debris from the vesicle suspension obtained in step e);
g) separating the vesicle suspension into populations of vesicles; and
h) providing vesicles from a population of vesicles which is characterized by the following parameters: (i) having a single predominant protein revealed after Coomassie staining an SDS PAGE that has an apparent molecular weight of 55 to 60 kDa, (ii) having a spherical appearance in electron micrographs and (iii) exhibiting a homogenous surface structure in electron micrographs.

2. The method of claim 1, wherein said method further comprises after step c) and prior to step d) the steps of:
treating said cells in deionized water solution; and
isolating the cellular membranes from said solution.

3. The method of claim 1, wherein said removing aggregated membranous debris from the vesicle suspension in step f) is carried out by filtration using a 0.45 uM filter.

4. The method of claim 1, wherein said method further comprises introducing a cargo agent of interest into the vesicles provided in step h).

5. The method of claim 4, wherein said introducing comprises the steps of:
a) suspending the vesicles in transfection buffer comprising an excess of cargo agent of interest;
b) carrying out electroporation; and
c) purifying loaded vesicles after electroporation.

6. The method of claim 4, wherein said cargo agent of interest is selected from the group consisting of: small molecule drugs, peptides, proteins, and nucleic acid molecules.

7. The method of claim 1, wherein said method further comprises sortagging a targeting compound to the sortaggable VSG on the vesicles provided in step h).

8. The method of claim 7, wherein said sortagging comprises the steps of:
a) treating the vesicles by sortase in the presence of targeting compound; and
b) purifying vesicles sortagged with the targeting compound.

9. The method of claim 7, wherein said targeting compound is an antibody or nanobody recognizing a target molecule on a target cell.

10. A vesicle comprising a VSG, preferably, a sortaggable VSG characterized by the following parameters: (i) having a single predominant protein revealed after Coomassie staining an SDS PAGE that has an apparent molecular weight of 55 to 60 kDa, (ii) having a spherical appearance in electron micrographs and (iii) exhibiting a homgeous surface structure in electron micrographs.

11. The vesicle of claim 10, which is loaded with a cargo agent of interest, preferably, selected from the group consisting of: small molecule drugs, peptides, proteins, and nucleic acid molecules.

12. The vesicle of claim 10, wherein said vesicle is sortagged with a targeting compound, preferably, an antibody or nanobody recognizing a target molecule on a target cell.

13. A method for treating and/or preventing a disease or medical condition comprising administering a vesicle as defined in claim 10.

14. (canceled)

15. A kit for carrying out the method of claim 1 comprising recombinant Trypanosoma brucei cells expressing a sortaggable VSG and at least one agent for carrying out the method as defined in claim 1.

16. The method of claim 1, wherein said population of vesicles is further characterized by an average diameter within the range of about 50 nm to about 500 nm, preferably, about 150 nm to about 250 nm, as determined by dynamic light scattering analysis.

17. The method of claim 1, wherein said recombinant Trypanosoma brucei cells lack GPI phospholipase C.

18. The method of claim 1, wherein said at least one protease inhibitor is the HALT protease inhibitor composition.

19. The vesicle of claim 10, wherein said vesicle is further characterized by an average diameter within the range of about 50 nm to about 500 nm, preferably, about 150 nm to about 250 nm, as determined by dynamic light scattering analysis.

20. The vesicle of claim 10, wherein said nucleic acid molecule is an antisense oligonucleotide or an expression construct encoding it.

21. The vesicle of claim 20, wherein said antisense oligonucleotide is suitable for RNA editing, preferably, for the generation of a neoepitope in an immunogenic peptide in a cancer cell.
